(19) United States
(12) Patent Application Publication
(10) Pub. No.: US 2017/0059595 A1
(43) Pub. Date: Mar. 2, 2017

(54) HIGH THROUGHPUT BIOCHEMICAL FLUOROMETRIC METHOD FOR MEASURING HDL REDOX ACTIVITY

(71) Applicant: THE REGENTS OF THE UNIVERSITY OF CALIFORNIA, Oakland, CA (US)

(72) Inventors: Theodoros Kelesidis, Los Angeles, CA (US); Otto O. Yang, Los Angeles, CA (US); Srinivasa T. Reddy, Cerritos, CA (US)

(21) Appl. No.: 15/120,727

(22) PCT Filed: Feb. 27, 2015
(86) PCT No.: PCT/US2015/018147

§ 371 Date: Aug. 22, 2016

Related U.S. Application Data
(60) Provisional application No. 61/946,441, filed on Feb. 28, 2014.

(51) Int. Cl. G01N 33/92; G06F 19/00; G01N 21/64

(52) U.S. Cl. CPC G01N 33/92; G01N 21/6428; G06F 19/322; G01N 2333/908; G01N 2333/904; G01N 2800/323; G01N 2800/50

(57) ABSTRACT

In various embodiments, a new, robust fluorometric cell-free biochemical assay that measures HDL redox activity (HRA) is provided. In certain embodiments the assay is based on the oxidation of the fluorochrome AMPLEX® RED in the presence of HRP. HRA correlated with previously validated cell-based (r=0.47, p<0.001) and cell-free assays (r=0.46, p<0.001). HRA measurement identified samples with dysfunctional HDL in established animal models of atherosclerosis and Human Immunodeficiency Virus (HIV) patients. Using an immunoaffinity method for capturing HDL the utility of this novel assay for measuring HRA in a high throughput format was demonstrated. HRA measurements correlated significantly with measures of cardiovascular disease such as carotid intima media thickness (r=0.35, p<0.01) and subendocardial viability ratio (r=−0.21, p=0.05) and physiological parameters such as metabolic and anthropometric parameters (p<0.05). This new fluorometric method that offers a reproducible and rapid means for determining HDL function/quality that is suitable for high throughput implementation.

### CROSS-REFERENCE TO RELATED APPLICATIONS

This application claims benefit of and priority to U.S. Ser. No. 61/946,441, filed Feb. 28, 2014, which is incorporated herein by reference in its entirety for all purposes.

### STATEMENT OF GOVERNMENTAL SUPPORT

This invention was made with Government support under Grant Nos: AI028697, DK090406, HL082823, HL095126, HL095132, HL105188, awarded by the National Institutes of Health. The Government has certain rights in the invention.

### BACKGROUND

There is a continuing search for new biomarkers of atherosclerotic disease. Manipulation of HDL has great potential in reducing cardiovascular risk; however, studies in humans suggest a complex relationship between HDL and atherosclerosis (Navab et al. (2011) Nat. Rev. Cardiol. 8(4): 222-232; Navab et al. (2009) J. Lipid. Res. 2009; 50 Suppl: S145-S149). Lower HDL values are not uniformly associated with excess cardiovascular risk while higher HDL levels may not always confer a protective benefit (Id.). Measuring HDL cholesterol levels provides information about the size of the HDL pool, but does not predict HDL composition or function. Thus, HDL function rather than absolute level may be a more accurate indicator of cardiovascular risk (Id.).

Due to the complexity of the HDL particles, measurement of HDL function has not been studied extensively in humans (Khera et al. (2011) N. Engl. J. Med. 364(2): 127-135; Patel et al. (2011) J. Am. Coll. Cardiol. 58(20): 2068-2075). Robust assays to evaluate the function of HDL are needed to supplement the measurement of HDL cholesterol levels in the clinic. Currently, HDL functional properties are most often determined by cell-based assays including the measurement of cholesterol efflux capacity (Patel et al. (2009) J. Am. Coll. Cardiol. 53(11): 962-971; Undurti et al. (2009) J. Biol. Chem. 284(45): 30825-30835; Van Lenten et al. (2007) J. Lipid. Res. 48(11): 2344-2353) Watson et al. (2011) J. Lipid. Res. 52(2): 361-373). However, several limitations render these cell-based assays inaccessible to many researchers and difficult to scale up for large-scale clinical trials and/or routine clinical use (Movva and Rader (2008) Clin. Chem. 54(5): 788-800). Along these lines, we previously developed a cell-free assay that measures HDL function by testing the effect of HDL on the production of reactive oxygen species (ROS) after oxidation and conversion of dichlorodihydrofluorescein diacetate (DCFH-DA) to fluorescent DCF (2′,7′-dichlorofluorescein) (Navab et al. (2001) J. Lipid. Res. 42(8): 1308-1317). Although this assay has been used in several studies in humans to study HDL function (Khera et al. (2011) N. Engl. J. Med. 364(2): 127-135; Patel et al. (2011) J. Am. Coll. Cardiol. 58(20): 2068-2075; Watanabe et al. (2009) J. Biol. Chem. 284(27): 18292-18301; Morgantini et al. (2011) Diabetes, 60(10): 2617-2623; Imaizumi et al. (2010) Drug Metab. Lett. 4(3): 139-148; Charles-Schoeman et al. (2009) Arthritis Rheum. 60(10): 2870-2879; Watanabe et al. (2007) J. Biol. Chem. 282(32): 23698-23707; Watanabe et al. (2012) Arthritis Rheum. 64(6): 1828-1837) it has not found widespread usage due to the oxidative instability of DCFH-DA. HDL oxidation may contribute to the formation of dysfunctional HDL (Navab et al. (2004) J. Lipid. Res. 45(6): 993-1007; Navab et al. (2006) Nat. Clin. Pract. Endocrinol. Metab. 2(9): 504-511) and we have previously shown that the oxidative properties of HDL are closely associated with HDL function (Kelesidis et al. (2011) J. Lipid. Res. 52(12): 2341-2351).

An alternative biochemical assay to quantify the HDL redox activity (HRA) that measures the products of redox cycling as the rate of oxidation of the fluorogenic probe dihydrorhodamine-123 (DHR) to fluorescent rhodamine 123 (Id.) was developed. However, despite the relative stability of DHR in contrast to DCF, it was determined that the complexity of the matrix-lipid-probe-ROS interactions in the setting of systemic inflammation may complicate interpretation of the results using the DHR assay (Kelesidis et al. (2011) J. Lipid. Res. 52(12): 2341-2351; Kelesidis et al. (2012) Lipids Health Dis. 11: 87).

### SUMMARY

In various embodiments, a new, robust, cell-free biochemical assay that measures HDL redox activity (HRA) is provided. In certain embodiments the assay is based on the oxidation of the fluorochrome AMPLEX® RED or AMPLEX® ULTRARED in the presence of HRP. It is noted that the AMPLEX® RED reagent and its variants are described in U.S. Pat. No. 4,384,042, which is incorporated herein by reference for the reagents described therein, and AMPLEX® ULTRARED and its variants are described in WO/2005/042504 (PCT/US2004/036546) which is incorporated herein by reference for the reagents described therein.

The HRA measured using the assays described herein correlated well with previously validated cell-based (r=0.47, p<0.001) and cell-free assays (r=0.46, p<0.001). HRA measurement identified samples with dysfunctional HDL in established animal models of atherosclerosis and Human Immunodeficiency Virus (HIV) patients. Using an immunoaffinity method for capturing HDL the utility of this novel assay for measuring HRA in a high throughput format was demonstrated. HRA measurements correlated significantly with measures of cardiovascular disease such as carotid intima media thickness and subendocardial viability ratio and physiological parameters such as metabolic and anthropometric parameters. The new assays described herein offers a reproducible and rapid means for determining HDL function/quality that is suitable for high throughput implementation

Accordingly, in various aspects, the invention(s) contemplated herein may include, but need not be limited to, any one or more of the following embodiments:

### Embodiment 1

A method of evaluating HDL function, said method including: contacting a sample including HDL with 10-acetyl-3,7-dihydroxyphenoxazine (AMPLEX® RED) or with AMPLEX® ULTRARED in the presence of horse radish peroxidase (HRP) in a reaction mixture to provide a measure of the endogenous hydroperoxide content of said HDL, wherein said hydroperoxide content is a measure of HDL redox activity (HRA) for the HDL in said sample and where elevated HRA is an indicator of dysfunctional HDL.

### Embodiment 2

The method of embodiment 1, wherein an elevated HRA is an HRA at least about 5% higher, or at least about 10% higher, or at least about 15% higher, or at least about 20% higher, or at least about 30% higher, or at least about 40% higher, or at least about 50% higher, or at least double or at least about 2.5 times higher, or at least about 3 times higher, or at least about 4 times higher, or at least about 5 times higher, or at least about 10 times higher than the HRA measured for a negative control, or than the HRA corresponding to an inflammatory index of 1, or than the HRA for samples (e.g., pooled samples) from healthy subjects.

### Embodiment 3

The method according to any one of embodiments 1-2, wherein said sample is contacted with 10-acetyl-3,7-dihydroxyphenoxazine (AMPLEX® RED).

### Embodiment 4

The method according to any one of embodiments 1-2, wherein said sample is contacted with AMPLEX® ULTRARED.

### Embodiment 5

The method according to any one of embodiments of embodiments 1-4, wherein said reaction mixture does not contain cholesterol oxidase.

### Embodiment 6

The method according to any one of embodiments 1-5, wherein cholesterol esterase is added to the reaction mixture so that peroxidation of HDL cholesterol in the form of cholesteryl esters versus free cholesterol can be determined.

### Embodiment 7

The method according to any one of embodiments of embodiments 1-6, wherein said detecting includes detecting the fluorescence or change in florescence of said reaction mixture over a time interval of at least 5 minutes, or at least 10 minutes, or at least 15 minutes, or at least 30 minutes, or at least 45 minutes, or at least 1 hour.

### Embodiment 8

The method of embodiment 7, wherein said fluorescence is quantified relative to the maximum fluorescence observed over the entire time interval.

### Embodiment 9

The method according to any one of embodiments 7-8, wherein said fluorescence is quantified relative to the corresponding time point of a reference control sample.

### Embodiment 10

The method according to any one of embodiments 7-9, wherein said detecting includes determining the concentration of the oxidation product of said AMPLEX® RED® from a standard curve for the oxidation product at the same time-point.

### Embodiment 11

The method of embodiment 10, said determining the concentration from a standard curve is performed using regression analysis.

### Embodiment 12

The method according to any one of embodiments 1-11, wherein said detecting includes determining the mean fluorescence readout (slope) for said reaction mixture and normalizing the value by the HDL concentration of said sample.

### Embodiment 13

The method according to any one of embodiments 1-12, wherein said sample is, or is derived from, non EDTA plasma.

### Embodiment 14

The method according to any one of embodiments 1-12, wherein said sample is, or is derived from, serum.

### Embodiment 15

The method of embodiment 14, wherein said sample is, or is derived from, apoB depleted serum.

### Embodiment 16

The method according to any one of embodiments 1-15, wherein said sample is, or is derived from a fresh (unfrozen) sample.

### Embodiment 17

The method according to any one of embodiments 1-15, wherein said sample is, or is derived from a cyropreserved sample.

### Embodiment 18

The method according to any one of embodiments 1-17, wherein said sample includes isolated HDL.

### Embodiment 19

The method of embodiment 14, wherein said sample includes HDL isolated by a method selected from the group consisting of ultracentrifugation, PEG precipitation, heparin MnCl₂ precipitation, sodium phosphotungstate precipitation, dextran sulfate precipitation, and immunoaffinity capture.

### Embodiment 20

The method of embodiment 14, wherein said sample includes HDL isolated by PEG precipitation.

### Embodiment 21

The method of embodiment 14, wherein said sample includes HDL isolated by immunoaffinity capture.

### Embodiment 22

The method according to any one of embodiments 1-21, wherein said detecting includes comparing, or normalizing, said measurement to a control.

### Embodiment 23

The method of embodiment 22, wherein said control includes a sample (e.g., a pooled sample) from healthy subjects.

### Embodiment 24

The method according to any one of embodiments 22-23, wherein said control includes a positive control.

### Embodiment 25

The method of embodiment 24, wherein said positive control includes a hydrogen peroxide (H₂O₂) working solution.

### Embodiment 26

The method according to any one of embodiments 22-25, wherein said detecting includes determining the difference between said measurement and the same measurement made for a negative control.

### Embodiment 27

The method of embodiment 26, wherein said negative control includes a reaction mixture without cholesterol.

### Embodiment 28

The method according to any one of embodiments 1-27, wherein said detecting includes determining the production of hydroxyradicals as a result of air oxidation of buffer based on the readout of a blank well that contains AMPLEX® RED® and subtracting the value from the fluorescent readout of test samples.

### Embodiment 29

The method according to any one of embodiments 1-28, wherein said method is performed in a high throughput format.

### Embodiment 30

The method according to any one of embodiments 1-29, wherein said method is performed in a multi-well plate.

### Embodiment 31

The method according to any one of embodiments 1-29, wherein said method is performed in a microfluidic device.

### Embodiment 32

The method of embodiment 31, wherein said method is performed in a droplet-based (segmented flow) microfluidic system.

### Embodiment 33

The method according to any one of embodiments 1-32, wherein elevated HRA is an HRA greater than the HRA measured for HDL from a normal healthy subject of the same age and gender.

### Embodiment 34

The method according to any one of embodiments 1-32, wherein elevated HR is an HRA greater than the HRA associated with an inflammatory index greater than 1.

### Embodiment 35

A method of determining the presence or risk of atherosclerosis in a subject, said method including: determining HDL redox activity (HRA) for HDL in a sample from said subject according to the method of any one of embodiments 1-34, wherein an elevated HRA as compared to that for a normal healthy subject indicates that said subject has or is at risk for atherosclerosis.

### Embodiment 36

The method of embodiment 35, wherein the elevated HRA and/or a diagnosis based, at least in part, on said level is recorded in a patient medical record.

### Embodiment 37

The method of embodiment 36, wherein said patient medical record is maintained by a laboratory, physician's office, a hospital, a health maintenance organization, an insurance company, or a personal medical record website.

### Embodiment 38

The method according to any one of embodiments 35-37, wherein a diagnosis, based at least in part on the HRA level is recorded on or in a medic alert article selected from a card, worn article, or radiofrequency identification (RFID) tag.

### Embodiment 39

The method according to any one of embodiments 35-38, wherein said HRA levels and/or a diagnosis based upon the HRA levels is recorded on a non-transient computer readable medium.

### Embodiment 40

The method according to any one of embodiments 35-39, wherein the HRA level is determined as part of a differential diagnosis.

### Embodiment 41

The method according to any one of embodiments 35-40, wherein said subject is a non-human mammal.

### Embodiment 42

The method according to any one of embodiments 35-40, wherein said subject is a human.

### Embodiment 43

A method for the treatment or prophylaxis of atherosclerosis, said method including: identifying a subject that has an elevated HDL redox activity as compared to a normal healthy individual or population or as compared to the same individual at an earlier time, where said elevated HDL redox activity is determined by the method of any one of embodiments 1-34; and performing further testing and/or treating said subject as a subject having or at elevated risk for atherosclerosis.

### Embodiment 44

The method of embodiment 43, wherein said subject is prescribed an additional test and/or said additional tests are performed.

### Embodiment 45

The method of embodiment 44, wherein said additional tests comprise one or more tests selected from the group consisting of blood tests for heart tissue damage or high risk for heart attack, electrocardiogram, stress test, coronary MRI, and coronary angiography.

### Embodiment 46

The method of embodiment 45, wherein said additional test includes a blood test selected from the group consisting of troponin I, T-00745, creatine phosphokinase (CPK), LDL, AST, ALT, and myoglobin.

### Embodiment 47

The method of embodiment 45, wherein additional test comprise a stress test selected from the group consisting of an exercise tolerance test, a nuclear stress test, cardiac MRI stress, and a stress echocardiogram.

### Embodiment 48

The method according to any one of embodiments 43-47, wherein said subject is prescribed a treatment and/or treated.

### Embodiment 49

The method of embodiment 48, wherein said treatment includes administration of a pharmaceutical.

### Embodiment 50

The method of embodiment 49, wherein said pharmaceutical includes one or more pharmaceuticals selected from the group consisting of a statin, a beta blocker, nitroglycerin or other nitrate, heparin, ACE inhibitor, angiotensin receptor blockers (ARB), aspirin and other anti-platelets, calcium channel blocker, and Ranolazine.

### Embodiment 51

The method according to any one of embodiments 48-50, wherein said treatment is a treatment selected from the group consisting of angioplasty, percutaneous intervention (PCI) including implantation of a stent, and coronary bypass surgery.

### Embodiment 52

A kit for performing a method of evaluating HDL function, said kit including: a container containing AMPLEX® RED® or AMPLEX® ULTRARED®; and a container containing one or more reagents for isolating HDL.

### Embodiment 53

The kit of embodiment 52, wherein said one or more reagents for isolating HDL comprise a reagent selected from the group consisting of PEG, heparin MnCL2, sodium phosphotungstate, dextran sulfate, and an antibody for immunoaffinity capture of HDL.

### Embodiment 54

The kit of embodiment 52, wherein said one or more reagents for isolating HDL comprise an antibody for immunoaffinity capture of HDL.

### Embodiment 55

The kit of embodiment 54, wherein said antibody is attached to a solid support.

### Embodiment 56

A method of screening for an agent that improves HDL function, said method including: contacting HDL with one or more test agents; and determining the HLD redox activity of said HDL according to the method of any one of embodiments 1-34, where a decrease in the HRA of said HDL, or the prevention of an increase in the HRA of said HDL indicates that said one or more test agents improve HDL function.

### Embodiment 57

The method of embodiment 56, wherein said contacting is ex vivo.

### Embodiment 58

The method of embodiment 56, wherein said contacting includes administering said one or more test agents to a mammal.

### DEFINITIONS

The term “low density lipoprotein” or “LDL” is defined in accordance with common usage of those of skill in the art. Generally, LDL refers to the lipid-protein complex which when isolated by ultracentrifugation is found in the density range d=1.019 to d=1.063.

The term “high density lipoprotein” or “HDL” is defined in accordance with common usage of those of skill in the art. Generally “HDL” refers to a lipid-protein complex which when isolated by ultracentrifugation is found in the density range of d=1.063 to d=1.21.

The term “HDL component” refers to a component (e.g. molecules) that comprises a high density lipoprotein (HDL). Illustrative components can include, but are not limited to apo A-I, paraoxonase, platelet activating factor acetylhydrolase, etc.

The term “test agent” refers to an agent that is to be screened in one or more of the assays described herein. The agent can be virtually any chemical compound. It can exist as a single isolated compound or can be a member of a chemical (e.g. combinatorial) library. In a certain embodiments, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Illustrative small organic molecules range in size up to about 5000 Da, more preferably up to 2000 Da, and most preferably up to about 1000 Da.

### BRIEF DESCRIPTION OF THE DRAWINGS

FIG. 1 illustrates the principle of the AMPLEX® RED assay of HDL function. 1: The acute-phase (AP) reaction favors the formation of dysfunctional HDL. In the basal state, HDL contains apoA-I and apoJ as well as 4 enzymes, paraoxonase (PON) and platelet-activating factor acetylhydrolase (PAF-AH), lecithin:cholesterol acyltransferase (LCAT), and plasma reduced glutathione selenoperoxidase (GSH peroxidase) that can prevent the formation of or inactivate the LDL-derived oxidized phospholipids found in oxidized LDL. As a result, in the basal state, HDL may be considered anti-oxidant. 2. As previously published (Navab M et al. (2001) Arterioscler. Thromb. Vasc. Biol., 21: 481-488), during the acute-phase reaction, A-I may be displaced by the pro-oxidant acute-phase reactant Serum amyloid A (SAA). Another pro-oxidant acute-phase reactant, ceruloplasmin, associates with HDL as does the anti-oxidant acute phase reactant apoJ. PON, PAF-AH, and LCAT decrease in HDL during the acute-phase reaction, and the lipid hydroperoxides LOOH) 5-hydroperoxyeicosatetraenoic acid (HPETE), hydroperoxyoctadecadienoic acid (HPODE), and cholesteryl linoleate hydroperoxide (CE-OOH) increase in HDL. The net effect of the changes in HDL during the acute-phase reaction is the production of pro-oxidant, HDL particles (AP-HDL or dysfunctional HDL). 3. HDL can be isolated using different methods such as ultracentrifugation, PEG precipitation and immunoaffinity capture (shown). Using immunoaffinity capture of HDL and commercially available antibodies against total human HDL, HDL is isolated from a specific volume (e.g. 100 μl) of either a) non EDTA plasma b) serum or c) apoB depleted serum 4. AMPLEX® RED (N-acetyl-3, 7-dihydroxyphenoxazine) reagent is a colorless substrate that reacts with hydrogen peroxide (H₂O₂) and more specifically with the OH⁻ radical in the presence of horseradish peroxidase (HRP) with a 1:1 stoichiometry to produce highly fluorescent resorufin (excitation/emission maxima=570/585 nm). This highly stable, sensitive and specific fluorogenic substrate for HRP has been widely used to develop a variety of fluorogenic assays for enzymes that produce hydrogen peroxide. For example AMPLEX® RED reagent coupled with the enzymes cholesterol oxidase and HRP permit the ultrasensitive quantitation of HDL cholesterol based on lipid peroxidation. Resorufin is produced by the reaction of the AMPLEX® RED reagent with H₂O₂ produced from the cholesterol oxidase-catalyzed oxidation of cholesterol. In the absence of cholesterol oxidase, the “endogenous” hydroperoxide content of a specific amount of HDL cholesterol can be quantified in the presence of HRP and AMPLEX® RED. High hydroperoxide content of a specific amount of HDL cholesterol has previously been shown to be significantly associated with abnormal HDL function. The background production of hydroxyradicals as a result of air oxidation of the buffer (based on the readout of the blank well that contains AMPLEX® RED reagent and buffer) is subtracted from the fluorescent readout of each well.

FIGS. 2A-2B illustrate the oxidation of AMPLEX® RED and effect of added HDL. In a 96 well flat bottom, 50 μl of 1× reaction buffer (0.5 M potassium phosphate, pH 7.4, 0.25 M NaCl, 25 mM cholic acid, 0.5% Triton® X-100) was added to each well alone or with 5 μg (cholesterol) of apoB depleted serum (as determined by a cholesterol assay) from a donor with anti-inflammatory HDL (HDL) and from a donor with acute phase HDL (AP-HDL), each in quadruplicates. 50 μl of HRP was then added to all wells followed by incubation at 37° C. for 60 min. 50 μl of AMPLEX® RED Reagent (final concentration 300 μM) was then added to each well for a total volume of 150 μl. Resorufin is produced by the reaction of the AMPLEX® RED reagent with H₂O₂ and the rate of production of resorufin was followed at 37° C. in one-minute intervals using a fluorescence microplate reader set to detect 530/590 nm excitation/emission. FIG. 2A: The means and standard deviations of the quadruplicate fluorescence measurements are plotted over time. FIG. 2B: The rates of change in fluorescence between 0 and 60 minutes (calculated by linear regression) are plotted for the quadruplicates, as well as means/standard deviations. The background fluorescence of the blank well (no HDL) was subtracted from the readout of each well for each time point.

FIGS. 3A-3B show that the AMPLEX® RED assay of HDL function can detect established effects of statins on functional properties of HDL in animal models of atherosclerosis. FIG. 3A: By using FPLC, HDL was isolated from three pooled plasma samples from LDLR^{-/-} mice on Western diet (LDLR^{-/-} WD) for two weeks and from three pooled plasma samples from LDLR^{-/-} mice on Western diet for two weeks that were also treated with pravastatin 12.5 μg/ml for two weeks. Each plasma sample was pooled from 4 mice (12 mice in total). Oxidation of AMPLEX® RED was assessed as in FIG. 2, using 2.5 μg (cholesterol) of added HDL. The oxidation slope of AMPLEX® RED in the presence of HDL from LDLR^{-/-} WD+ statin was normalized to the oxidation slope of AMPLEX® RED in the presence of HDL from LDLR^{-/-} WD, and the percent relative differences are shown. The data represent the average of measurements from three independent experiments. There was a statistically significant reduction in the oxidation slope of AMPLEX® RED in the presence of HDL isolated from LDLR^{-/-} WD+ statin mice compared with the oxidation slope of DHR in the presence of HDL isolated from LDLR^{-/-} WD mice (**P=0.01). FIG. 3B: By using FPLC, HDL was isolated from three pooled plasma samples from ApoE^{-/-} female mice on Western diet (ApoE^{-/-} WD) for two weeks and from three pooled plasma samples from ApoE^{-/-} female mice on Western diet for two weeks that were also treated with pravastatin 12.5 μg/ml for two weeks. Each plasma sample was pooled from 4 mice (12 mice in total). Oxidation of AMPLEX® RED was assessed as in FIG. 3A. There was a statistically significant reduction in the oxidation slope of AMPLEX® RED in the presence of HDL isolated from ApoE^{-/-} WD+ statin mice compared with the oxidation slope of AMPLEX® RED in the presence of HDL isolated from ApoE^{-/-} WD mice (**P=0.01).

FIG. 4 shows that the AMPLEX® RED assay of HDL function can detect acute phase HDL in vivo in subjects previously shown to have dysfunctional HDL. ApoB depleted serum was isolated by PEG precipitation from 50 healthy subjects and 100 patients with HIV infection and that have previously been shown to have acute phase HDL (Kelesidis et al. (2012) Lipids Health Dis. 11: 87). The AMPLEX® RED oxidation rate (AROR) as a marker of HDL redox activity (HRA) was determined as described in FIG. 2 and FIG. 20. The HIV-infected subjects had significantly higher HRA (1.59±0.53) compared to the uninfected subjects 1.01±0.31) (p<0.001)

FIG. 5 shows that the readout from the AMPLEX® RED Assay of HDL function correlates significantly to the readout of a previously validated cell based assay of HDL function. Thirty samples of FPLC-purified HDL were assessed for their HDL redox activity (HRA) using the AMPLEX® RED assay as shown in FIG. 2, and their HDL inflammatory index was determined in a cell-based assay as described in Materials and Methods. The values from each assay are plotted against each other.

FIG. 6 shows that the readout from the AMPLEX® RED Assay of HDL function correlates significantly to the readout of a previously validated biochemical cell free assay of HDL function. ApoB depleted serum was isolated by PEG precipitation from 50 healthy subjects and 100 patients with HIV infection that have previously been shown to have acute phase HDL (Kelesidis et al. (2012) Lipids Health Dis. 11: 87). HDL redox activity (HRA) was determined with the AMPLEX® RED assay as described in FIG. 2 and with the dihydrorhodamine (DHR) assay as described in Methods. Non cryopreserved apoB depleted serum was used for the DHR assay and the readout was normalized by the readout of a pooled control as described in 18. The values from each assay are plotted against each other.

FIG. 7 shows that the AMPLEX® RED assay of HDL function in combination with immunoaffinity capture of HDL can detect acute phase HDL in vivo in subjects previously shown to have dysfunctional HDL. HDL was isolated using immunoaffinity capture as described in Methods from 30 healthy subjects and 30 patients with HIV infection that have previously been shown to have acute phase HDL (Kelesidis et al. supra.). The following different matrices were added in 96 well plates for immunoaffinity capture of HDL: a) purified HDL isolated by ultracentrifugation (5 μg of HDL cholesterol as determined by cholesterol assay), b) apo-B depleted serum (5 μg of HDL cholesterol as determined by cholesterol assay) c) apo-B depleted serum (100 μl) d) plasma (100 μl). In the latter two methods, the fluorescent readout (that corresponds to HRA) was normalized to the HDL cholesterol concentration (measured by the clinical lab). ApoB depleted serum and plasma was isolated by PEG precipitation and HDL was also isolated by ultracentrifugation as described in methods. The AMPLEX® RED oxidation rate (AROR) as a marker of HDL redox activity (HRA) was determined as described in FIG. 2 and FIG. 20. The HIV-infected subjects had significantly higher HRA (A: 1.66±0.37; B: 1.54±0.32; C: 1.40±0.33; D: 1.32±0.32) compared to the uninfected subjects (A: 1.05±0.28; B: 0.95±0.23; C: 0.81±0.24; D: 0.73±0.24) (p<0.01 for all comparisons)

FIG. 8 shows that the use of different commercially available antibodies does not affect significantly the immunoaffinity capture of HDL and determination of HRA using the AMPLEX® RED assay. HDL was isolated using immunoaffinity capture as described in Methods and FIG. 7 from 30 healthy subjects (white circles) and 30 patients with HIV infection (solid circles). Two different antibodies were used (kit A and Kit B) as described in Methods. The AMPLEX® RED oxidation rate (AROR) as a marker of HRA was determined as described in FIG. 2 and FIG. 20. The values from each assay are plotted against each other.

FIG. 9 shows that increased HDL redox activity (HRA), as measured by the AMPLEX® RED method and the immunoaffinity capture, is independently associated with progression of atherosclerosis in HIV-1-infected subjects in vivo. Scatter plot of the Rate of Change in Carotid intima-media thickness (CIMT) (ΔCIMT) and HRA for 55 HIV-infected subjects (solid circles) and 36 uninfected controls (white circles). HDL ELISA kit was used to capture HDL in 96-well plates (kit B) as described in Methods. HRA was determined as described in FIG. 2 and FIG. 20. The values from HRA for each subject are plotted against ΔCIMT. In multivariate analysis of the HIV-infected subjects, higher baseline HRA was associated with the ΔCIMT increasing by 2.3 mm/yr (95% CI=(0.24, 5.6); p=0.03) but no association between ΔCIMT and HRA was seen in the controls (not shown).

FIG. 10 shows that the AMPLEX® RED assay of HDL function can detect previously established favorable effects of exercise on HDL function. HRA was measured as described in FIG. 2 and FIG. 20 in a cohort of 90 humans looking into the effect of exercise on metabolic and other physiological parameters. In this study we found that high-intensity resistance training (RT) improved central and brachial blood pressures in the overweight untrained (OU) group, while having no effect on major indices of arterial stiffness in overweight/obese young men, without weight loss (unpublished data). Using the samples from this study we found that HRA was significantly lower in both trained groups compared to the untrained group (LT vs. OU: 0.65±0.12 vs. 0.91±0.17, p=<0.001; OT vs. OU: 0.68±0.11 vs. 0.91±0.17, p=0.003), and LT and OT were not significantly different (p=0.12).

FIG. 11, panels A-D, shows that the HRA as measured with the novel assay is significantly associated with numerous anthropometric, metabolic and physiological parameters in humans. HRA was measured as described in FIG. 2 and FIG. 20 in a previous cohort of 100 humans looking into the effect of exercise on metabolic and other physiological parameters. The values from HRA for each subject are plotted against representative physiological parameters such as Body Mass Index (BMI) (panel A), subendocardial viability ratio (SEVR) (panel B), a noninvasive measure of subendocardial perfusion, C reactive protein (CRP) (panel C), and oxidized Low Density Lipoprotein (ox-LDL) (panel D).

FIG. 12 shows that increasing amounts of HRP can increase the efficiency of detection of hydroperoxides carried by a specific amount of HDL cholesterol. In a 96 well flat bottom plate, 50 μl of 1× reaction buffer was added to each well alone or with 5 μg (cholesterol) of apoB depleted serum (as determined by a cholesterol assay) from a donor with anti-inflammatory HDL (HDL) and from a donor with acute phase HDL (AP-HDL), each in quadruplicates. 50 μl of HRP (0.5-4 U/ml) was then added to all wells followed by incubation at 37° C. for 60 min. 50 μl of AMPLEX® RED Reagent (final concentration 300 μM) was then added to each well for a total volume of 150 μl and the rate of production of resorufin was followed at 37° C. in one-minute intervals for 60 minutes. The rates of change in fluorescence between 0 and 60 minutes are plotted for the quadruplicates, as well as means/standard deviations. In the absence of HRP, fluorescence quenching and lipid-probe interactions lead to reduction in the fluorescence readout after addition of a specific amount of HDL cholesterol compared to the fluorochrome alone, consistent with our previous observations with other fluorochromes such as DHR and DCF. Addition of ≧2 U/ml of HRP lead to a specific amplification of the quantification of the hydroperoxides associated with a specific amount of HDL cholesterol. A representative sample from each type of HDL (HDL vs AP-HDL) is shown and similar results were observed for 5 different other samples (5 HDL and 5 AP-HDL). In addition similar results with HRP were observed when 5 μg of HDL cholesterol isolated by FPLC or ultracentrifugation were added to the reaction (data not shown).

FIG. 13 shows that the AMPLEX® RED assay can detect a concentration dependent increase in the amount of hydroperoxides associated with increasing amount of added HDL cholesterol. HDL isolated by ultracentrifugation was added in varying concentrations (cholesterol) to 300 μM AMPLEX® RED in a 96 well flat bottom plate and the rate of change in fluorescence was measured as in FIG. 2 in the presence of 4 U/ml of HRP. The rates of change in fluorescence (means and standard deviations) are plotted against the amounts of added HDL. In addition similar results with HRP were observed when HDL cholesterol isolated by PEG precipitation was added to the reaction (data not shown). There was a concentration dependent increase in the fluorescent readout with increasing amount of added HDL cholesterol in the presence of HRP in contrast to a concentration dependent decrease in the readout with increasing amount of added HDL cholesterol with other fluorescent probes (DCF and DHR).

FIG. 14 shows that the AMPLEX® RED Assay can reliably quantify the content of hydroperoxides associated with a specific amount of HDL cholesterol when ≦10 μg of HDL is added. HDL was isolated by ultracentrifugation from 3 HIV infected patients known to have acute phase HDL (AP-HDL) and 3 patients with normal HDL (as determined using a previous assay of HDL function (Kelesidis et al. (2011) J. Lipid. Res. 52(12): 2341-2351). HDL was then added in varying concentrations (cholesterol) to 300 μM AMPLEX® RED in a 96 well flat bottom plate and the rate of change in fluorescence was measured as in FIG. 2 in the presence of 4 U/ml of HRP. The rates of change in fluorescence were normalized against the added HDL cholesterol amount (in μg of cholesterol as determined by a cholesterol assay) and are plotted (means and standard deviations) against the amounts of added HDL. Similar results were observed when HDL cholesterol isolated by PEG precipitation was added to the reaction (data not shown).

FIG. 15 illustrates a correlation of effect of HDL on AMPLEX® RED oxidation using different methods of HDL isolation. HDL was isolated by ultracentrifugation or PEG precipitation from 5 HIV infected patients known to have acute phase HDL (AP-HDL; shown in solid black circles) and 5 patients with normal HDL (shown in white circles). 5 μg of HDL cholesterol was then added to 300 μM AMPLEX® RED in a 96 well flat bottom plate and the rate of change in fluorescence was measured as in FIG. 2 in the presence of 4 U/ml of HRP. The mean rates of change in fluorescence are plotted.

FIG. 16 illustrates low inter-assay variability between measurements of HDL effects. Rate of oxidation of AMPLEX® RED in the presence of six different samples of HDL isolated by PEG precipitation from 6 subjects (3 HIV infected subjects with AP-HDL and 3 healthy subjects with normal HDL) was assessed as described in FIG. 2, using 5 μg (cholesterol) of added HDL. The data (means of quadruplicates) from four independent experiments are plotted. The mean inter-assay variability for these six samples was 8.6% (range 4.9 to 9.7%), and the mean intra-assay variability was 4.6% (range 2.9-7.2%). When HDL from the same subjects was isolated by UC (not plotted) the mean inter-assay variability for these six samples was 10.6% (range 6.9 to 12.6%), and the mean intra-assay variability was 6.8% (range 4.8-9.1%). In addition, measurement of the rate of oxidation of AMPLEX® RED over 60 min lead to reduced experimental variability compared to endpoint measurement of fluorescence at 60 min (data not shown).

FIG. 17 shows that commercially available resorufin standards can be used to standardize fluorescence-based quantification of the hydroperoxide content of a specific amount of HDL cholesterol. A commercially available resorufin fluorescence reference standard can be used to prepare a standard curve to determine the moles of fluorescent product produced in the AMPLEX® RED reaction according to the manufacturer's instructions. Endpoint measurement of the fluorescence signal that corresponds to production of resorufin and oxidation of the AMPLEX® RED reagent was performed at 60 minutes as described in FIG. 2. The reference 2 mM resorufin standard was diluted accordingly to generate a standard curve of resorufin that would “fit” the dynamic range of the measured fluorescence at 60 minutes for the specific assay. Towards this end, the amount of the added cholesterol and the time of the reaction for certain photomultiplier sensitivity needs to be titrated carefully. The triplicate fluorescence readings for each standard were averaged and the mean fluorescence was calculated. The average fluorescence of the blank sample (AMPLEX® RED alone without HDL) was subtracted from all the standards and samples and the adjusted fluorescence was calculated. The adjusted fluorescence of the standards was plotted as a function of the concentration of the resorufin standards. An example of a standard curve with a dynamic range 15.625-500 nM and six standards is shown. The fluorescence of the HDL samples was calculated in the presence of 5 μg (cholesterol) of added HDL. HDL was isolated by PEG precipitation from HIV infected subjects with acute phase HDL (AP-HDL) and healthy subjects with normal HDL. The means of quadruplicates were calculated (adjusted fluorescence). The amount of produced resorufin for each HDL sample was calculated using the equation obtained from the linear regression of the standard curve substituting adjusted fluorescence values for each sample. 2 representative samples (one with normal HDL [dashed line] and one with AP-HDL [solid line]) are shown. Since the resorufin standards are run in parallel with the samples over 60 minutes the slope of change in fluorescence per min (FU/min) can be converted into the slope of nM of resorufin produced per min (nM/min). Thus, using this approach, the resorufin concentration that is produced as a result of the specific oxidation of the AMPLEX® RED Reagent by the hydroperoxides present in a specific amount of each HDL cholesterol sample, can be measured and can be used as a surrogate measure of the HDL redox activity and HDL function.

FIG. 18 illustrates the fluorescence readout of the AMPLEX® RED assay of HDL function can be normalized against the readout of a specific amount of HDL cholesterol isolated from pooled apoB depleted serum of healthy subjects A) The AMPLEX® RED oxidation rate (AROR) was determined as described in FIG. 2 after adding 5 μg of apoB depleted serum (isolated by PEG precipitation) from 50 cryopreserved serum blood bank specimens from healthy subjects. The values represent means of triplicate samples. There was an approximately 3-fold difference between the lowest and highest AROR value (median 155, IQR 119-180 FU/min; range 74-246 FU/min). B) The 50 HDL samples were pooled in groups of five samples (pentads; 0.5 μg of HDL cholesterol from each sample) so that the total amount of HDL cholesterol in each pooled sample (pentad) was 5 μg, for a total of 10 pentads. Then the pentads were combined in various combinations and different number (5, 10, 15, 20, 25, 30, 35, 40, 45, 50) of HDL samples so that the total amount of HDL cholesterol at each pooled sample would be 5 μg. The AROR was determined as described in FIG. 2. The values represent means of triplicate samples. Using this methodology there was approximately a 2-fold reduction in the variability of determination of the AROR (for the same amount of HDL cholesterol) in healthy subjects (median 153, IQR 141-166 FU/min; range 120-186 FU/min). Thus, to correct for inter-assay variability across different plates and to standardize the assay, a pooled HDL control sample from at least 30 healthy volunteers may be included in each plate, AROR can be determined and sample fluorescence values may be normalized by this pooled value. The individual normalized AROR (nAROR) [nAROR=(AROR/AROR control] is evaluated as a ratio to the AROR of a control HDL isolated from the pooled serum. Thus, using this method we avoid expression of results of AROR in arbitrary units (FU/min) and results between different studies can be comparable provided the same pooled control is used.

FIG. 19 shows that a specific amount of HDL cholesterol isolated from pooled blood bank specimens of healthy subjects can be used as a universal control to standardize the AMPLEX® RED assay of HDL function. HDL was isolated using PEG precipitation from 3 different groups (A, B, C; each 30 samples) of cryopreserved serum blood bank specimens. The HDL samples in each group were pooled as described in FIG. 17 (three different blood bank pools). The AMPLEX® RED oxidation rate (AROR) was determined as described in Materials and Methods. The mean AROR among the 3 different blood bank pools was comparable. Thus, this current approach may be used to create a universal control for determination of DOR by combining HDL samples from at least 30 different donors.

FIG. 20 shows that the HDL concentration as determined by the clinical laboratory can be used to adjust the fluorescence readout for the amount of HDL cholesterol in each sample in the AMPLEX® RED assay of HDL function. ApoB depleted serum was isolated by PEG precipitation from 20 subjects (10 healthy and 10 with HIV infection and acute phase HDL). The AMPLEX® RED oxidation rate (AROR) was determined as described in FIG. 2 and HDL was added using two different methods (A and B). In method A the HDL cholesterol concentration of each sample was determined using a cholesterol assay as described in the Methods section and then 5 μg of HDL cholesterol was added to each well. The individual normalized AROR (nAROR) [nAROR=(AROR/AROR control] is a measure of the HDL redox activity (HRA) and is evaluated as a ratio to the AROR of a control HDL isolated from pooled serum as described in FIG. 18. In Method B the HDL cholesterol concentration of each sample (mg/dl) was measured by the clinical lab and this value is routinely available in the setting of standard clinical care. A specific volume of apoB depleted serum (50 μl) was added to each well, the AROR for each sample was determined as above and this readout was normalized by the HDL cholesterol concentration of each sample (n_{HDL}AROR). A control HDL sample was created after pooling equal volumes of apoB depleted serum from 30 healthy blood bank serum. The HDL concentration of this pooled HDL control was calculated from the HDL concentrations of the individual samples (measured in mg/dl by the clinical lab) and the fluorescence readout was normalized by this value (n_{HDL}AROR control). The individual normalized to control AROR is evaluated as a ratio to the AROR of a control HDL isolated from pooled serum [nAROR=(n_{HDL}AROR/n_{HDL}AROR control]. The values represent means of triplicate samples and the correlation coefficient is shown. Data from healthy subjects are shown as white circles and data from HIV infected subjects are shown as gray circles.

FIG. 21 shows that the standardization method with the pooled control minimizes the effect of multiple freeze-thaw cycles on determination of HDL redox activity (HRA) using the AMPLEX® RED assay. Oxidation rate of AMPLEX® RED (AROR) in the presence of 20 different samples of HDL isolated by PEG precipitation from heparin plasma [10 from patients with Human Immunodeficiency virus (HIV-1) infection and 10 from healthy volunteers (Non HIV)] was assessed as described in Methods and in FIG. 2, using 2.5 μg (cholesterol) of added HDL. The AROR of each sample was determined within 6 hours after collection of the blood specimen and after 1-5 freeze-thaw cycles. The values represent means of triplicate samples. The % relative HRA of each HDL sample after each extra freeze-thaw cycle (for up to 5 cycles) was significantly higher (paired t-test p<0.05 for all datapoints) compared to the HDL sample that was isolated within 6 hours. Although the HRA values tended to significantly increase after each extra freeze-thaw, their correlations with the HRA value from the HDL sample that was isolated within 6 hours remained statistically significant (p<0.05 for all data points; data not shown). In addition, the individual normalized AROR was evaluated as a ratio to the AROR of a control HDL isolated from pooled serum as described in FIGS. 18 and 20. The control HDL matched the freeze-thaw cycles of the respective HDL samples (for example if the samples were thawed once the pooled control was made from HDL samples that were thawed once, etc.). This standardization method improved the correlations of the relative HRA values with the HRA value from the HDL sample that was isolated within 6 hours (data not shown) and tended to minimize the effect of multiple freeze-thaw cycles on determination of HRA.

FIG. 22 shows that the standardization method minimizes the effect of different matrices on oxidative properties of HDL. Oxidation rate of AMPLEX® RED (AROR) in the presence of 13 different samples of HDL [7 from patients with Human Immunodeficiency virus (HIV-1) infection and 6 from healthy volunteers (Non HIV)] isolated by PEG precipitation from heparin plasma, citrate plasma and serum was assessed as described in Methods and in FIG. 2. The values represent means of all the samples. The HRA values from plasma citrate samples correlated significantly (p<0.01) with the HRA values from serum samples but heparin interfered with the readout (data not shown). In addition, the HRA of each sample was normalized by the HRA value of the control sample and the % relative HRA was determined as in FIG. 10. The suggested standardization method improved the correlations of the HRA values (data not shown) and tended to minimize the effect of different matrices on determination of HRA.

FIG. 23 shows that long term storage of blood specimens tends to increase HDL redox activity (HRA) as determined by the AMPLEX® RED assay but the results are comparable between different time points. The Multicenter AIDS Cohort Study (MACS) has defined a group of men who remained HIV-1-seronegative despite hundreds to thousands of high-risk sexual exposures in the 1980s. The MACS cohort recruited men in 1985 for natural history studies (Kaslow et al. (1987) Am. J. Epidemiol. 126: 310-318), and has continued to follow subjects every 6 months to the present. Using 9 stored serum samples from this cohort that were stored for 27 and 28 years at −80° C., we determined the effect of long term storage at −80 C. on HDL redox activity (HRA) as described in FIG. 2. The readout of each sample was expressed as % relative to the average readout of all 9 samples at 27 years of cryopreservation. The HRA as determined by the AMPLEX® RED assay significantly increased after cryopreservation for one extra year (125±41% vs 100±31%, p value for paired t test=0.04) and the readouts from the 2 groups correlated significantly (r=0.69, p<0.01).

FIG. 24 shows that HDL isolated using immunoaffinity capture of HDL is largely free of albumin. 50 μl of plasma (n=10) was added into 96 wells and was isolated using immunoaffinity capture of HDL according to the manufacturer's instructions (Kit A). After 5 washes, 300 μl of albumin bromocresol green reaction (BCG) reagent (Thermo Scientific Inc.) were added to each well and after 90 second incubation at 37° C. the optical density at 630 nm was read. Results are expressed as % relative value compared to the positive control (50 μl of plasma). The median relative albumin content bound to HDL was 0.90% with the BCG method, one of the most sensitive and specific methods to detect albumin. The minimal detection of HDL-bound albumin (<0.5% relative to the positive control) was also confirmed with a secondary antibody against albumin conjugated to horseradish peroxidase (HRP) (Pierce Inc.) (data not shown). Similar results were obtained with Kit B.

### DETAILED DESCRIPTION

In various embodiments, rapid, reproducible and inexpensive methods are provided to measure functional properties of High Density Lipoprotein (HDL), e.g., in a biological sample. Cardiovascular disease (CVD), principally heart disease and stroke, is the Nation's leading killer for both men and women among all racial and ethnic groups. High-density lipoprotein (HDL) is a major carrier of cholesterol in the blood. HDL and cardiovascular disease show an inverse correlation. However, recent studies indicate that higher HDL levels may not always be protective and can become dysfunctional losing their cardioprotective effects. HDL particles can vary in size, density, composition, and functional properties influencing their association with atherosclerosis. Further, emerging evidence suggests that HDL function is not always accurately predicted by HDL cholesterol levels.

The laboratory assessment of HDL function remains in its infancy. In vitro assays of HDL function have been developed by various research laboratories but are laborious, nonstandardized, and poorly validated with regard to human outcomes. Robust laboratory assays of HDL functions and validation of the usefulness of these assays for predicting cardiovascular risk and assessing response to therapeutic interventions are critically important and of great interest to cardiovascular clinicians and investigators and clinical chemists.

We previously developed a fluorometric biochemical assay based on the effect of HDL on the oxidation of the fluorochrome dihydrorhodamine 123 (DHR). This cell-free assay assesses the oxidative potential of HDL using the measurement of fluorescence due to DHR oxidation over time and distinguishes the oxidative potential of HDL taken from different persons. The details of this method have been described in a publication (Kelesidis et al. (2011) J. Lipid Res. 52(12): 2341-2351). However, particularly when this method was used with cryopreserved samples, it was found that there are biochemical interactions that appear to limit the utility of this assay to the research setting. In addition the previous method of HDL isolation described did not allow high throughput isolation of purified HDL (not contaminated with other proteins e.g., albumin). Finally the biochemical mechanism of the interaction of the previous fluorochrome (DHR) with reactive oxygen species has not been entirely elucidated and appears to provide unreliable results in a number of contexts.

To address these limitations a novel approach was used to quantify reactive oxygen species (ROS) based on a fluorochrome called AMPLEX® RED. This fluorochrome has well characterized biochemistry and using enzymatic amplification of ROS quantification in combination with purification of HDL, particularly in combination with immunoaffinity capture of HDL, a novel biochemical assay is provided that measures redox (functional) properties of HDL. The results of this assay are highly reproducible even with cryopreserved samples. Moreover the assay is amendable to a multi-well (e.g., high throughput) format. This new method offers an inexpensive, accurate, and rapid means for determination of oxidative properties of HDL that can be applied easily to large scale clinical studies and thus has numerous potential commercial applications.

Surprisingly it was demonstrated that this new approach, particularly in combination with immunoaffinity capture of HDL for HDL isolation, overcomes many confounding factors that affected the readout in previously described cell free assays of HDL function and provides a measurement of HRA that correlates well with previously validated cell-based and cell-free assays of HDL function. Because a large proportion of cholesterol in blood is in the form of cholesteryl esters, cholesterol esterase can be used to produce free cholesterol from cholesterol esters (Amundson et al. (1999) J. Biochem. Biophys. Meth. 38(1): 43-52; Mishin et al. (2010) Free Radic. Biol. Med. 48(11): 1485-1491). In addition the assay may be modified and cholesterol esterase may or may not be added in the AMPLEX® RED reagent so that peroxidation of HDL cholesterol in the form of cholesteryl esters versus free cholesterol can be determined (Id.).

In the absence of cholesterol oxidase the AMPLEX® RED detects the intrinsic hydroperoxide content of a specific amount of HDL cholesterol. Moreover, the use of immunoaffinity capture (among other HDL purification methods) allows HDL isolation and use of this method in large scale studies and removal of much of the albumin bound to the HDL particle that may alter the association of ROS with lipoproteins (Kelesidis et al. (2011) J. Lipid. Res. 52(12): 2341-2351). Finally, the inter-assay variability of the new assay of <15% compares favorably with cell-based assays of HDL function, that typically have variability of >15% (Roche et al. (2008) FEBS Lett. 582(13): 1783-1787).

In addition using animal models of atherosclerosis and two different human studies it was demonstrated that this new assay can be used as a marker of cardiovascular disease and biologic processes in vivo. The correlation of HRA as measured by the new assays described herein to the biologic readout of HDL in a cell-based assay is consistent with a proposed mechanism whereby HDL exerts its effects through modulating ROS (Navab et al. (2004) J. Lipid. Res. 45(6): 993-1007; Navab et al. (2006) Nat. Clin. Pract. Endocrinol. Metab. 2(9): 504-511), and indicates that the assay accurately reflects HDL function. Further support for the biological relevance of this measurement is the finding that for the same amount of HDL cholesterol the HRA was significantly reduced in HDL from statin treated mice with atherosclerosis compared to HDL from non-statin treated mice.

Treatment of these mice with statins has previously been shown to reduce inflammatory properties of HDL (Navab et al. (2001) J. Lipid. Res. 42(8): 1308-1317) and it is also demonstrated that the new assays described herein can detect the favorable effect of statins on functional properties of HDL. Moreover, the assays can detect dysfunctional HDL in patients with HIV infection.

The fact that the new assay quantifies HRA and that the HRA levels correlate with surrogate measures of cardiovascular disease and other physiological parameters in humans such as obesity and insulin resistance increases its applicability to biological samples, at least in the context of cardiovascular diseases. Without being bound to a particular theory, it is believed that this is because 1e-oxidants (i.e., hydroxyl radical) contribute to oxidative modifications taking place in the affected arterial wall during atherogenesis (Kelesidis et al. (2011) J. Lipid. Res. 52(12): 2341-2351; Kelesidis et al. (2012) Lipids Health Dis. 11: 87; Kelesidis et al. (2011) Lipids Health Dis. 10: 35). Thus, while “oxidized HDL” is not a chemically defined term, the oxidation rate of the indicator(s) used in the present assays (e.g., AMPLEX® RED, AMPLEX® ULTRARED, etc.) corresponds to the intrinsic HRA of specific amount of HDL lipoproteins.

It was additionally a surprising discovery that matrix effects, freeze thaw, sample handling and long term storage of blood specimens at −8° C. can affect the HRA but the relative differences in HRA between different samples can still be reliably detected with the present assays, in either serum or non EDTA plasma, fresh or cryopreserved samples, and with up to 2 freeze-thaw cycles.

Thus, the new assays described herein offer a rapid method for measuring the redox properties of HDL. They yield results that correlate well with previously validated cell-based and cell-free assays of HDL function and can be used as a marker of cardiovascular disease and biologic processes in humans. This new technical approach offers a convenient tool for studies of the role of HDL functional phenotype in the development of atherosclerosis in vivo.

### HRA Assays.

In certain illustrative, but non-limiting embodiments, assays that provide a measure of HDL redox activity HRA) are provided. In certain embodiments the assays involve contacting a sample (e.g., a biological sample) comprising HDL with 10-acetyl-3,7-dihydroxyphenoxazine (AMPLEX® RED) in the presence of horse radish peroxidase in the reaction mixture to provide a measure of the hydroperoxide content (e.g., endogenous hydroperoxides content) of the HDL, where the hydroperoxide content is a measure of HDL redox activity (HRA) for the HDL in the sample and where elevated HRA is an indicator of dysfunctional HDL.

Assays using AMPLEX® RED can be read out fluorometrically or spectrophotometrically using standard methods well known to those of skill in art. Instructions for such measurements are also provided by the manufacturer of the reagents.

In certain embodiments the assay mixture does not contain cholesterol oxidase and thereby provides a clear measure of the “endogenous” hydroperoxides content of the HDL being assays. In addition, or alternatively, in some embodiments, cholesterol esterase is added to (e.g., provided in) the reaction mixture so that peroxidation of HDL cholesterol in the form of cholesteryl esters versus free cholesterol can be determined.

In various embodiments, the detecting/quantification of the assay reaction comprises detecting the fluorescence or change in florescence or the absorbance or change in absorbance of the reaction mixture over a time interval of at least 1 minute, or at least 5 minutes, or at least 10 minutes, or at least 15 minutes, or at least 30 minutes, or at least 45 minutes, or at least 1 hour, or at least 1.25 hrs, or at least 1.5 hrs. In certain embodiments the fluorescence is quantified relative to the maximum fluorescence observed over the entire time interval. In certain embodiments the fluorescence (or absorbance) is quantified relative to the corresponding time point of a reference control sample. In certain embodiments the detecting comprises determining the concentration of the oxidation product of the AMPLEX® RED® from a standard curve for the oxidation product at the same timepoint (e.g., using a regression analysis). In certain embodiments the detecting comprises determining the mean fluorescence (or absorbance) readout (slope) for the reaction mixture and, optionally, normalizing the value by the HDL concentration of the sample.

Any of a number of biological samples containing HDL can be assayed using these methods. In certain embodiments the sample comprises or is derived from whole blood or a blood fraction. In certain embodiments the sample is, or is derived from, non EDTA plasma. In certain embodiments the sample is, or is derived from, serum (e.g., apoB depleted serum). In certain embodiments the sample is, or is derived from a cryopreserved sample (e.g., a sample that has been cryopreserved for at least 1 hour, or at least 12 hours, or at least 24 hours, or at least 2 days, or at least 1 week, or at least 2 weeks, or at least 1 month, or at least 6 months, or at least 12 months, or longer).

In certain embodiments the assay is performed HDL that has been isolated from or purified from a biological sample. Methods of isolating HDL are well known to those of skill in the art. Illustrative isolation methods include, but are not limited to ultracentrifugation, PEG precipitation, heparin MnCl₂ precipitation, sodium phosphotungstate precipitation, dextran sulfate precipitation, and immunoaffinity capture. Protocols for these and other HDL isolation methods are readily available. Thus, for example, illustrative, but non-limiting protocols for HDL isolation by PEG precipitation, heparin MnCl₂ precipitation, sodium phosphotungstate precipitation, and dextran sulfate precipitation are described by Wieve and Smith (1985) Clin. Chem., 31(5): 746-750). Similarly, illustrative, but non-limiting methods of immunoaffinity capture of HDL are described by Watanabe et al. (2012) Arthritis Rheum., 64(6): 1828-1837, and kits for HDL immunoaffinity capture are commercially available (see, e.g., Genway Inc., Biotang Inc., Cusabio Inc, WKEA Inc., etc.) and methods are illustrated herein in the Examples.

In certain embodiments the assays involve determining the difference between the fluorescence or absorption measurement and the same measurement made for a negative control (e.g., a reaction mixture lacking cholesterol). In certain embodiments the assays involve comparing, or normalizing, the measurement to a positive control (e.g., hydrogen peroxide (H₂O₂) working solution). In certain embodiments the assays involve determining the production of hydroxyradicals as a result of air oxidation of buffer based on the readout of a blank well that contains AMPLEX® RED® and subtracting the value from the fluorescent readout of test samples.

In various embodiments, illustrative, but non-limiting embodiments HRA is determined to be elevated when the measured HRA is greater than the HRA measured for HDL from a normal healthy subject (e.g., the same age and/or gender) and/or greater than the “normal healthy” HRA level determined for a population. In certain embodiments the HRA level is compared to the HRA level determined for the same subject at an earlier time point to determine the presence and/or progression of a pathology.

In certain embodiments the HRA measured in the assay is identified as elevated when it is an HRA greater than the HRA associated with (e.g., measured for subjects having) an HDL-inflammatory index greater than 1. Methods of determining HDL-inflammatory index are known to those of skill in the art (see, e.g., Watson et al. (2011) J. Lipid Res., 52(2): 361-373; Navab et al. (2001) J. Lipid Res. 42(8): 1308-1317 which are incorporated herein by reference for the inflammatory index assays described herein). In this assay an HDL-inflammatory index value >1.0 is considered pro-inflammatory and a value <1.0 is considered anti-inflammatory.

The assays described can readily be performed in a multi-well plate format (e.g., a 96 well, a 100 well, a 320 well, a 384 well, an 864 well, and a 1536 well format). The assays can also readily be implemented using microfluidic platforms (e.g., Lab-on-a-Chip devices). In certain illustrative, but non-limiting embodiments, the assays described herein are well suited for droplet-based (or segmented flow) microfluidic systems (see, e.g. Huebner et al. (2008) Lab on a Chip. 8: 1244; deMello (2006) Nature 442: 394). In illustrative, but non-limiting segmented flow system water-in-oil emulsions are made to spontaneously form in microfluidic channels as a result of capillary instabilities between the two immiscible phases. Microdroplets of precisely defined volumes and compositions can be generated at frequencies of several kHz. Furthermore, by encapsulating reagents of interest within isolated compartments separated by a continuous immiscible phase, both sample cross-talk and dispersion (diffusion- and Taylor-based) can be eliminated, which leads to minimal cross-contamination and the ability to time analytical processes with great accuracy. Additionally, since there is no contact between the contents of the droplets and the channel walls (which are wetted by the continuous phase) absorption and loss of reagents on the channel walls is prevented. Such systems have proven to be well suited to fluorescence detection methods (see, e.g., Casadevall (2011) J. Visualized Exper., 58: e3437).

As indicated above, the assays described herein are reproducible and offers an inexpensive, accurate, and rapid means for determination of oxidative properties of HDL. Because the assays measure a biochemical rather than biologic process, they are more precise than previous cell based assays that determine functional properties of HDL. Moreover the results correlate well with a validated cell-based assay. This new technical approach may offers a convenient tool for studies of the role of HDL functional phenotype in the development of atherosclerosis in vivo.

### Illustrative Uses.

In addition to research uses, it is believed the assays described herein find considerable utility in, inter alia, diagnostic/prognostic application, in methods of treatment, and for screening for therapeutic agents.

Thus, in certain embodiments, methods for the presence or risk of atherosclerosis in a subject where the method involves determining HDL redox activity (HRA) for HDL in a sample from the subject using the methods described herein, wherein an elevated HRA (e.g., as compared to that for a normal healthy subject, or as compared to the HRA associated with an inflammatory index greater than 1, etc.) indicates that the subject has or is at risk for atherosclerosis. In certain embodiments the elevated HRA and/or a diagnosis based, at least in part, on said level is recorded in a patient medical record (e.g., a medical record is maintained by a laboratory, physician's office, a hospital, a health maintenance organization, an insurance company, a personal medical record website, and the like). In certain embodiments the HRA level is recorded on or in a medic alert article (e.g., a card, worn article, radiofrequency identification (RFID) tag, and the like). In certain embodiments the HRA levels and/or a diagnosis based upon the HRA levels is recorded on a non-transient computer readable medium. In certain embodiments the HRA level is determined and/or recorded as part of a differential diagnosis. In various embodiments, the subject is a non-human mammal (e.g., veterinary uses are contemplated) and in certain embodiments, the subject is a human.

In certain illustrative, but non-limiting embodiments methods for treatment or prophylaxis of atherosclerosis, are provided where the methods involve identifying a subject that has an elevated HDL redox activity as compared to a normal healthy individual or population or as compared to the same individual at an earlier time, where said elevated HDL redox activity is determined by the methods described herein and performing further testing and/or treating the subject as a subject having or at elevated risk for atherosclerosis. In certain embodiments the subject is prescribed an additional test and/or the additional tests are performed. In certain embodiments illustrative, but non-limiting additional tests comprise one or more tests selected from the group consisting of blood tests for heart tissue damage or high risk for heart attack, electrocardiogram, stress test, coronary MRI, and coronary angiography. In certain illustrative, but non-limiting embodiments the additional test(s) comprises a blood test selected from the group consisting of troponin I, T-00745, creatine phosphokinase (CPK), LDL, AST, ALT, and myoglobin. In certain illustrative, but non-limiting embodiments the additional test(s) comprise a stress test selected from the group consisting of an exercise tolerance test, a nuclear stress test, cardiac MRI stress, and a stress echocardiogram. In certain illustrative, but non-limiting embodiments the subject is prescribed a treatment and/or treated. In certain illustrative, but non-limiting embodiments the treatment comprises administration of a pharmaceutical (e.g., a statin, a beta blocker, nitroglycerin or other nitrate, heparin, ACE inhibitor, angiotensin receptor blockers (ARB), aspirin and/or other anti-platelets factor, a calcium channel blocker, and Ranolazine). In certain illustrative, but non-limiting embodiments the treatment is a treatment selected from the group consisting of angioplasty, percutaneous intervention (PCI) including implantation of a stent, and coronary bypass surgery.

In certain illustrative, but non-limiting embodiments methods are provided for screening for an agent that improves HDL function where the methods involve contacting HDL with one or more test agents; and determining the HLD redox activity of the HDL according to a method described herein, where a decrease in the HRA of said HDL, or the prevention of an increase in the HRA of said HDL indicates that said one or more test agents improve HDL function. In certain illustrative, but non-limiting embodiments the contacting is ex vivo. In certain illustrative, but non-limiting embodiments the contacting comprises administering said one or more test agents to a mammal.

It will be recognized that these uses are illustrative and non-limiting. Numerous other uses will be recognized by one of skill in the art. For example, the risk for or progression of other pathologies characterized by oxidized lipid formation can be determined (e.g., in the context of a differential diagnosis). Illustrative pathologies include for example, celiac disease (see, e.g., Feretti (2012) J. Lipids, //dx.doi.org/10.1155/2012/587479), Parkinson's disease (see, e.g., Farooqui et al. (2011) Parkinson's Disease Article ID 247467), and the like.

Using the teaching provided herein, numerous other uses of the assays described herein will be available to one of skill in the art.

### EXAMPLES

The following examples are offered to illustrate, but not to limit the claimed invention.

### Example 1

### High Throughput Biochemical Fluorometric Method for Measuring HDL Redox Activity

In this example, a new assay to quantify HRA, a measure of HDL function, in a cell-free biochemical assay is described. AMPLEX® RED reagent in the absence of cholesterol oxidase and in the presence of horseradish peroxidase (HRP) specifically quantifies the endogenous lipid hydroperoxides of a specific amount of HDL cholesterol. We demonstrate that this approach, in combination with immunoaffinity capture of HDL for HDL isolation, overcomes the confounding factors that affected the readout in the previously described cell free assays of HDL function (Kelesidis et al. (2011) J. Lipid. Res. 52(12): 2341-2351; Kelesidis et al. (2012) Lipids Health Dis. 11: 87).

### Materials and Methods

Reagents:

Dihydrorhodamine 123 (DHR) was obtained from Molecular Probes (Eugene, Oreg.). DHR was prepared as concentrated stock of 50 mM in dimethyl sulfoxide (DMSO) as previously described (Kelesidis et al. (2011) J. Lipid. Res. 52(12): 2341-2351). Iron-free HEPES (N-2-hydroxyethylpiperazine-N′-2-ethanesulfonic acid)-buffered saline (HBS, HEPES 20 mM, NaCl 150 mM, pH 7.4) was prepared as previously described (Id.). The DHR stock was diluted 1:1000 in HEPES saline solution to prepare a working solution of 50 μM. Reagents from the AMPLEX® RED Cholesterol Assay Kit (Catalog number A12216, Life Technologies, Grand Island, N.Y.) were used for the new assay. These reagents included the AMPLEX® RED reagent (10-acetyl-3,7-dihydroxyphenoxazine), Hydrogen Peroxide (H₂O₂) working solution, Resorufin fluorescence reference standard, horseradish peroxidase (HRP), Cholesterol esterase, reaction buffer (0.5 M potassium phosphate, pH 7.4, 0.25 M NaCl, 25 mM cholic acid, 0.5% Triton® X-100). Pravastatin sodium (Lot No. M000301, Catalog number P6801) was purchased from LKT Laboratories, Inc.

HDL and LDL Purification:

HDL and LDL were isolated from cryopreserved human plasma (with or without added sucrose) by ultracentrifugation, fast performance liquid chromatography (FPLC), or precipitation with polyethylene glycol. These were aliquoted and stored as previously described (Kelesidis et al. (2011) J. Lipid. Res. 52(12): 2341-2351; Havel et al. (1955) J. Clin. Invest. 34(9): 1345-1353; Hedrick et al. (1993) J. Biol. Chem. 268(27): 20676-20682; Watson et al. (1995) J. Clin. Invest. 96(6): 2882-2891; Widhalm and Pakosta (1991) Clin. Chem. 37(2): 238-240).

Immunoaffinity Capture of HDL:

We have previously used immunoaffinity capture of HDL to study changes in the proteome associated with dysfunctional HDL (Watanabe et al. (2009) J. Biol. Chem. 284(27): 18292-18301; Watanabe et al. (2007) J. Biol. Chem. 282(32): 23698-23707; Watanabe et al. (2012) Arthritis Rheum. 64(6): 1828-1837). Briefly, 96-well polyvinyl chloride microfilter plates (BD Biosciences) were precoated with 1-5 μg/ml of chicken anti-human HDL antibodies (GenWay Biotech, San Diego, Calif.) overnight at 4° C. Following incubation of the pre-coated plates with individual plasma samples diluted at 1:10 with 1× PBS, serum at 1:20 dilution, or HDL at 1:2 dilution, the plates were washed thoroughly. Detection of HDL was confirmed by HDL-capturing sandwich enzyme-linked immunosorbent assays (ELISA) as described previously using corresponding primary antibodies to human ApoA-I at 1:2,500 dilution (Id.). In addition, total HDL was captured using polyclonal antibodies included in 5 commercially available kits [Genway Inc (kit A); Biotang Inc (kit B), Cusabio Inc (kit C), China; WKEA Inc, China (kit D); Wuhan EIAab., Ltd; China (kit E)) according to the manufacturers' instructions.

Measurement of HDL Cholesterol:

HDL cholesterol was quantified using a standard colorimetric assay (Thermo DMA Co., San Jose, Calif., USA) as previously described (Kelesidis et al. (2011) J. Lipid. Res. 52(12): 2341-2351). Samples were also assayed by the UCLA Clinical Laboratory for total cholesterol, high-density lipoprotein (HDL), non-HDL cholesterol and triglycerides (TG).

Measurement of Total HDL Protein:

The total HDL protein “captured” in each well was measured using the BCA Protein Assay as previously described (Watanabe et al. (2009) J. Biol. Chem. 284(27): 18292-18301; Charles-Schoeman et al. (2009) Arthritis Rheum. 60(10): 2870-2879; Watanabe et al. (2007) J. Biol. Chem. 282(32): 23698-23707; Watanabe et al. (2012) Arthritis Rheum. 64(6): 1828-1837). In addition, the total HDL protein concentration for each sample was measured using total HDL ELISA and the above commercially available kits (Kits A, B) according to the manufacturer's instructions.

Detection of Albumin:

50 μl of plasma (n=10) was added into 96 wells and was isolated using immunoaffinity capture of HDL according to the manufacturer's instructions (Kit A). After 5 washes, 300 μl of albumin bromocresol green reaction (BCG) reagent (Thermo Scientific Inc) were added to each well and after a 90 second incubation at 37° C. optical density was measured at 630 nm. Results are expressed as % relative value compared to the positive control (50 μl of plasma). Detection of albumin was also confirmed by albumin ELISA according to the manufacturer's instructions (Pierce) using corresponding antibodies to human albumin conjugated to HRP.

HDL Inflammatory Index:

The HDL-inflammatory index was determined for each subject's HDL as described previously (Watson et al. (2011) J. Lipid. Res. 52(2): 361-373; Navab et al. (2001) J. Lipid. Res. 42(8): 1308-1317). In this assay a value >1.0 is considered pro-inflammatory and a value <1.0 is considered anti-inflammatory.

DHR-Based Cell-Free Assay of HDL Function:

The DHR assay was performed as previously described (Kelesidis et al. (2011) J. Lipid. Res. 52(12): 2341-2351). Briefly, quadruplicates of HDL (5 μg of cholesterol unless otherwise specified were added to 96-well plates (polypropylene, flat bottom, black, Fisher Scientific, USA). HBS was added to each well to a final volume of 150 μl, followed by addition of 25 μl of the working solution of 50 μM DHR, for a total volume of 175 μl. Immediately following DHR addition, the plate was protected from light, placed in the fluorescence plate reader and fluorescence of each well was assessed at two minute intervals for an hour with a Synergy 2 Multi-Mode Microplate Reader (Biotek, Vermont, USA), using a 485/538 nm excitation/emission filter pair with the photomultiplier sensitivity set at medium. The oxidation rate was calculated for each well as the slope for the linear regression of fluorescence intensity over 60 minutes, expressed as FU minute-1 (fluorescence units per minute). HRA was calculated as the mean of quadruplicates for the wells containing the HDL sample.

AMPLEX® RED Assay:

Method A: Use of PEG Precipitation for HDL Isolation.

ApoB depleted serum was isolated from human plasma using PEG precipitation and then a specific amount of HDL cholesterol (5 μg; quantified using a standard colorimetric assay, Thermo, CA, USA) was added to 96-well plates (polypropylene, flat bottom, black, Fisher Scientific, USA) in quadruplicates with the appropriate volume of 1× reaction buffer for a total volume of 50 μl per reaction well. A 2 mM resorufin solution was used to prepare a standard curve to determine the moles of product produced in the AMPLEX® RED reaction. The appropriate amount of 2 mM resorufin reference standard was diluted into 1× reaction buffer to produce resorufin concentrations of 0 to ˜20 μM). 1× reaction buffer without cholesterol was used as a negative control. A 20 mM H₂O₂ working solution was used as a positive control. A volume of 50 μL was used for each reaction. 50 μL of 300 μM of AMPLEX® RED reagent (Invitrogen) containing 2 U/mL HRP, and 0.2 U/mL cholesterol esterase were then added to each microplate well containing the samples and controls. The fluorescence of each well was assessed at one-minute intervals over 60 minutes with a plate reader (Biotek, Vermont, USA), using a 530/590 nm filter pair. The fluorescence at each timepoint can be expressed as: 1) relative to the maximum fluorescence observed over 60 minutes 2) relative to fluorescence of the corresponding timepoint of a reference control sample 3) as specific concentration of resorufin (μM) extrapolated from the resorufin standard curve at the specific timepoint using 4 parameter logistic regression analysis. The slope of the reaction of the AMPLEX® RED reagent with the endogenous hydroperoxides present in HDL in the absence of cholesterol oxidase, corresponds to the endogenous HRA of each sample and was calculated over 60 min using the Gen5 2.01 software (Biotek, Vermont, USA). Alternatively, a specific volume (50 μl) of apoB-depleted serum was added in each well in quadruplicates and the mean fluorescence readout (slope) was normalized by the HDL cholesterol concentration of each sample as measured by the clinical lab (mg/dL).

Method B: Use of HDL Immunocapture for HDL Isolation.

The following matrices were used for HDL capture in 96 well plates: 1) specific amount of commercially available purified HDL (Sigma) (5 μg of HDL cholesterol) 2) specific amount of purified HDL isolated by ultracentrifugation (5 μg of HDL cholesterol) 3) specific amount of apo-B depleted serum isolated by PEG precipitation (5 μg of HDL cholesterol) 4) specific volume of plasma or serum (100 μl) 5) specific volume of apo-B depleted serum (100 μl). These matrices were then added into 96 wells and HDL was isolated using immunoaffinity capture of HDL as described in Methods. The AMPLEX® RED assay was performed as described above. When a specific volume (100 μl) of apoB-depleted serum or plasma/serum was added in each well in quadruplicates, the mean fluorescence readout (slope) was normalized by the HDL cholesterol concentration of each sample as measured by the clinical lab (mg/dL).

Human Subjects:

Subjects with Coronary Artery Disease (CAD)

Blood samples were collected from patients with coronary artery disease (CAD) or equivalent as defined by the National Cholesterol Education Program Adult Treatment Panel III criteria (Executive Summary of The Third Report of The National Cholesterol Education Program (NCEP) Expert Panel on Detection, Evaluation, And Treatment of High Blood Cholesterol In Adults (Adult Treatment Panel III). JAMA (2001) 285(19): 2486-2497) and were collected from patients referred to the cardiac catheterization laboratory at the Center for Health Sciences at the University of California, Los Angeles. After signing a consent form approved by the Human Research Subject Protection Committee of the University of California, Los Angeles, the patient donated a fasting blood sample collected in a heparinized tube. Plasma samples were also randomly selected from pre-treatment samples remaining from a previously described study in which all patients had coronary artery disease or equivalent (Watson et al. (2011) J. Lipid. Res. 52(2): 361-373). All of these patients were on a statin (Id.).

Human Immunodeficiency Virus (HIV-1)-Infected Individuals:

Fifty HIV-1-infected individuals on combination antiretroviral therapy (ART) with suppressed viremia (below 50 copies of RNA/ml) (48 males and 2 females; median age 44, range 18-53 years) were recruited at the University of California, Los Angeles (UCLA) as previously described (Id.). These patients had no documented coronary atherosclerosis and normal total cholesterol (200 mg/dl), LDL cholesterol (130 mg/dl), HDL cholesterol (males, >45 mg/dl; females, >50 mg/dl), and triglycerides (<150 mg/dl), were not receiving hypolipidemic medications and were not diabetic. Fifty normal volunteers (42 males and 8 females; median age 42, range 18-57 years), matched by age and gender, were recruited under a protocol approved by the Human Research Subject Protection Committee of the University of California, Los Angeles (UCLA IRB). Blood samples from a previously completed matched cohort study that was designed to investigate the role of ART therapy and HIV-1 infection on the risk for subclinical atherosclerosis and its progression were also used for validation of the new assay; the study design has been previously published (Kelesidis et al. (2012) J. Infect. Dis. 206(10): 1558-1567). In this cohort subjects were enrolled as risk factor (age, sex, race/ethnicity, smoking status, blood pressure, and menopause status)-matched triads of HIV-1-infected individuals with viremia <500 RNA copies/ml with (n=29) or without (n=26) use of protease inhibitor (PI) therapy, and HIV-1-uninfected subjects (n=36).

Healthy Subjects:

Blood bank specimens were collected from healthy young blood donors according to previously well-defined criteria (Boulton (2008) Transfus. Med. 18(1): 13-27; Price (2008) Standards for blood banks and transfusion services. 25th ed, Bethesda (Md.), American Association of Blood Banks) More specifically the donors were young (range 19-40 years old) had no known underlying diseases including diabetes, were known to have normal lipid profile and were not receiving hypolipidemic medications.

Exercise Study Participants:

In this cross sectional study, 90 young adult males, ages of 18-30 were recruited and categorized into 3 phenotypes based on training status and Body Mass Index (BMI): lean trained (LT, n=30, ≧4 d/wk resistance training (RT), BMI<25 kg/m2), overweight trained (OT, n=30, ≧4 d/wk RT, BMI>27 kg/m2) and overweight untrained populations (OU, n=30, no structured exercise regimen, BMI>27 kg/m²). Overweight untrained subjects participated in only light intensity physical activity ≦2 times/wk. Participants with overt chronic disease symptoms, as indicated by screening, comprehensive history and/or physical examination were excluded from the study. Potential participants who had documented cardiovascular disease or used tobacco products or medications that influence cardiovascular function, body composition or insulin indices in the prior 6 months were excluded from the study. All of the study protocols were approved by the UCLA IRB.

Mice:

ApoE and LDLR null mice originally purchased from the Jackson Laboratories on a C57BL/6J background were obtained from the breeding colony of the Department of Laboratory and Animal Medicine at the David Geffen School of Medicine at UCLA as previously described (Navab et al. (2005) Arterioscler. Thromb. Vasc. Biol. 25(7): 1426-1432). The mice were maintained on a Western diet (Teklad, Harlan, catalog # TD88137) for 2 weeks and a group of mice was also treated with pravastatin at 12 μg/ml drinking water, or approximately 50 μg per day for two weeks. All experiments were performed using protocols approved by the Animal Research Committee at UCLA.

Data Collection:

Metabolic syndrome was defined by National Cholesterol Education Program (NCEP) criteria (Executive Summary of The Third Report of The National Cholesterol Education Program (NCEP) Expert Panel on Detection, Evaluation, And Treatment of High Blood Cholesterol In Adults (Adult Treatment Panel III). JAMA (2001) 285(19): 2486-2497). In the HIV cohort fasting glucose, insulin, lipids, high-sensitivity C-reactive protein (hs-CRP), cardiovascular disease-related measurements, CD4⁺ T cell counts and HIV-1 RNA levels were previously determined (Kelesidis et al. (2012) J. Infect. Dis. 206(10): 1558-1567). Carotid artery intima-media thickness (CIMT) of the far wall of the right common carotid artery was measured at baseline and longitudinally as previously published (Id.). In the exercise study measurements of serum glucose, serum insulin, homeostasis model assessment for insulin resistance (HOMA), oxLDL, hs-CRP, body composition parameters, anthropometric parameters, blood pressure (e.g., brachial systolic and diastolic pressure, bSBP, bDBP) maximal strength testing, subendocardial viability ratio (SEVR) were performed as previously described (Roberts et al. (2013) Metabolism, 62(5): 725-733).

Statistical Analysis:

Statistical analyses were performed with the use of Stata statistical software 12 (StataCorp LP., College Station, Tex.). Group means were compared using the Student's t-test for unpaired variates with p<0.05 considered to be statistically significant. Correlation coefficients between variables were calculated using least squares linear regression. In the HIV cohort, conditional logistic regression modeling for matched pairs data stratified by triad evaluated associations with CIMT progression. Covariates significant in the univariate analysis (p<0.05) were examined together in multivariate analysis. In the exercise study, post-hoc Pearson correlation analyses were used to determine the relationships between HRA and cardiovascular and metabolic disease risk biomarkers.

### Results

AMPLEX® RED can Specifically Measure Lipid Peroxidation of a Specific Amount of HDL.

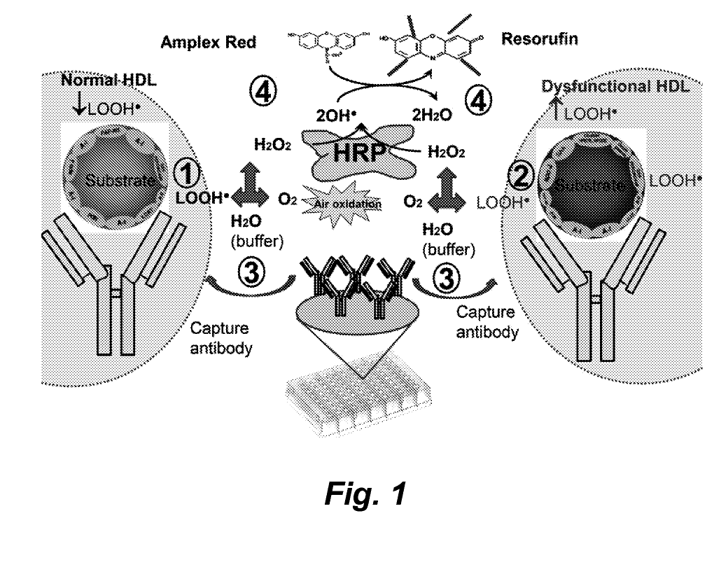

AMPLEX® RED in the presence of the enzyme cholesterol oxidase has been reliably used to quantify cholesterol content of HDL based on lipid peroxidation of HDL (Amundson et al. (1999) J. Biochem. Biophys. Meth. 38(1): 43-52; Mishin et al. (2010) Free Radic. Biol. Med. 48(11): 1485-1491). The biochemical reaction of the AMPLEX® RED Reagent with the OH radical in the presence of HRP to produce highly fluorescent resorufin and measure peroxides is well established (Id.). In the absence of cholesterol oxidase and in the presence of HRP AMPLEX® RED specifically quantifies the endogenous lipid hydroperoxides of a specific amount of HDL cholesterol (FIG. 1). Using this modification (no cholesterol oxidase), for the same amount of HDL cholesterol differences in the rate of lipid peroxidation between different samples (as detected by the AMPLEX® RED reagent) would correspond to differences in HDL redox activity (FIG. 1).

Lipid Probe Interactions are Still Present when Using AMPLEX® RED in Fluorescent Assays of HDL Function.

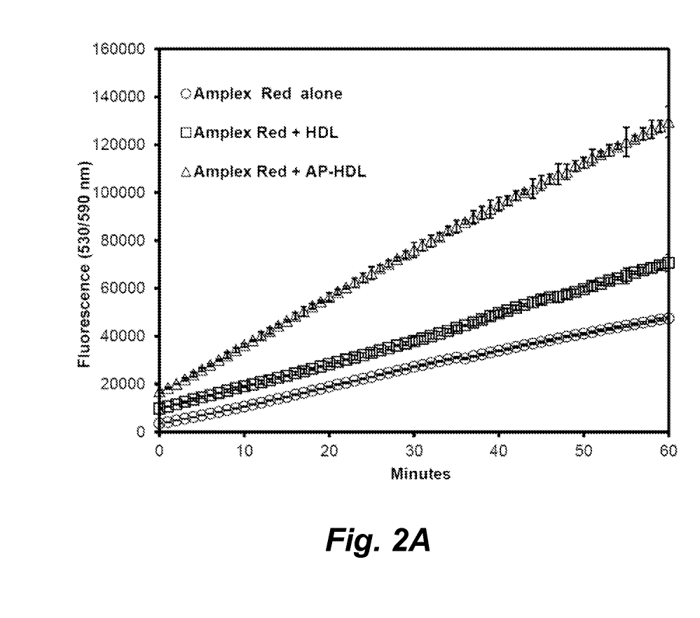

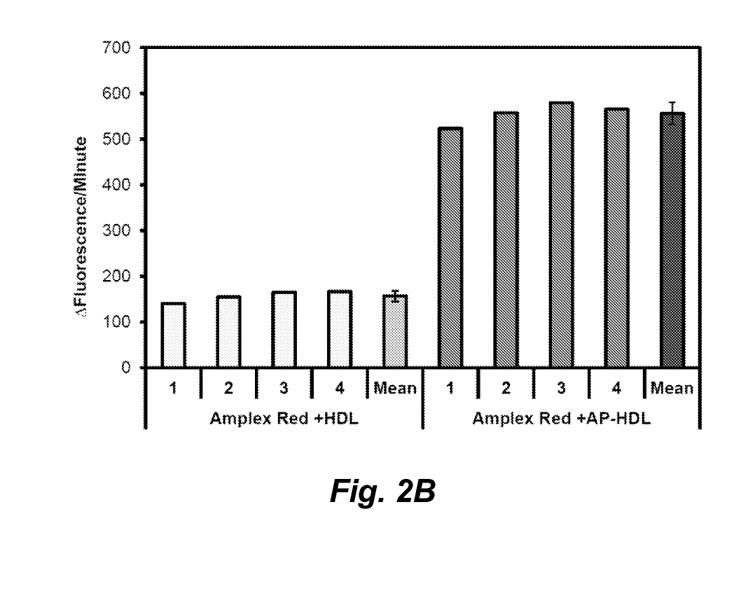

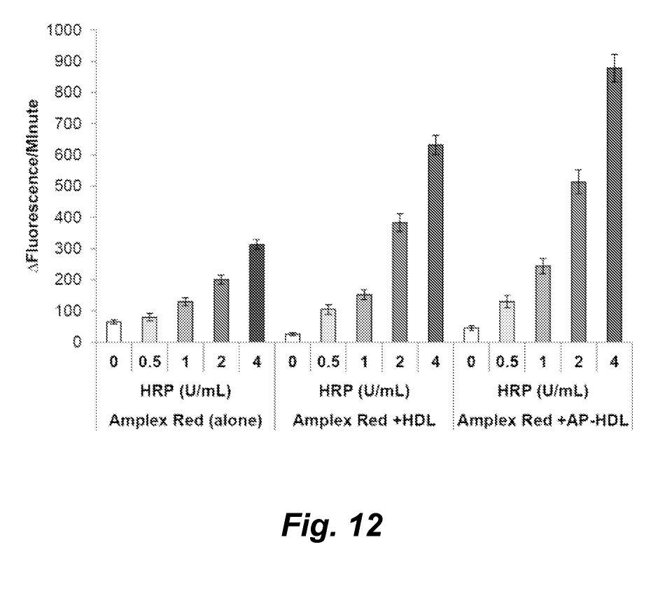

Since use of fluorescent assays of HDL function may allow study of the role of the functional phenotype of HDL in the setting of large scale studies (Kelesidis et al. (2011) J. Lipid. Res. 52(12): 2341-2351) especially with the use of a robust, reproducible, high throughput method to extract HDL from patient serum such as PEG precipitation (Patel et al. (2011) J. Am. Coll. Cardiol. 58(20): 2068-2075), we determined the (apo) B-depleted serum-AMPLEX® RED interactions in patients with inflammatory conditions such as HIV versus healthy subjects. Alone, AMPLEX® RED exposed to air became oxidized (and therefore fluorescent) at a linear rate within 60 minutes (FIG. 12, FIG. 2). The rate of oxidation of AMPLEX® RED was significantly less with added HDL, and different HDL samples (from patient with dysfunctional HDL compared to healthy subjects) showed clearly different effects in this regard (FIG. 12). Furthermore, when the amount of added HDL was varied, there was a clear dose-dependence in the oxidation rate of AMPLEX® RED that was linear in the range of 1.25 to 10 μg (cholesterol) of added HDL per well in the assay (data not shown). Thus, apoB depleted serum-probe interactions were also present with AMPLEX® RED similarly to the previously used fluorescent probes (Kelesidis et al. (2012) Lipids Health Dis. 11:87).

Use of AMPLEX® RED in Combination with HRP May Overcome Lipid Probe Interactions when Measuring Lipid Peroxidation of HDL.

To determine whether the reduction in the fluorescence signal of AMPLEX® RED after addition of HDL can be minimized or abolished by adding an enzyme that specifically catalyzes the lipid peroxidation and the oxidation of AMPLEX® RED (Gutheil et al. (2000) Anal. Biochem. 287(2): 196-202; Peus et al. (1999) J. Invest. Dermatol. 112(5): 751-756; Richer and Ford (2001) Mol. Hum. Reprod. 7(3): 237-244), we tested the effect of addition of different concentrations of HRP on the oxidation rate of AMPLEX® RED in the presence of a specific amount of HDL cholesterol. Increasing concentrations of HRP 1-10 U/ml increased the oxidation rate of AMPLEX® RED in the setting of lipid peroxidation of specific amount of HDL, compared to when AMPLEX® RED was used without any HRP (FIG. 12). Determination of the rate of production of resorufin was performed by measuring the slope of fluorescence increase during the first 60 minutes, when the rate of oxidation is linear (FIG. 2). These differences could be demonstrated using both purified HDL isolated by ultracentrifugation (data not shown) and apoB depleted serum (FIG. 2), a matrix that is characterized by more prominent lipid probe interactions (Kelesidis et al. (2012) Lipids Health Dis. 11: 87). Thus, the relative differences in HRA between different groups of samples (dysfunctional HDL versus normal HDL) can be quantified with the use of AMPLEX® RED and HRP with minimal lipid-probe interactions.

AMPLEX® RED can Reliably Measure Lipid Peroxidation of a Specific Amount of HDL and Determine HDL Redox Activity (HRA).

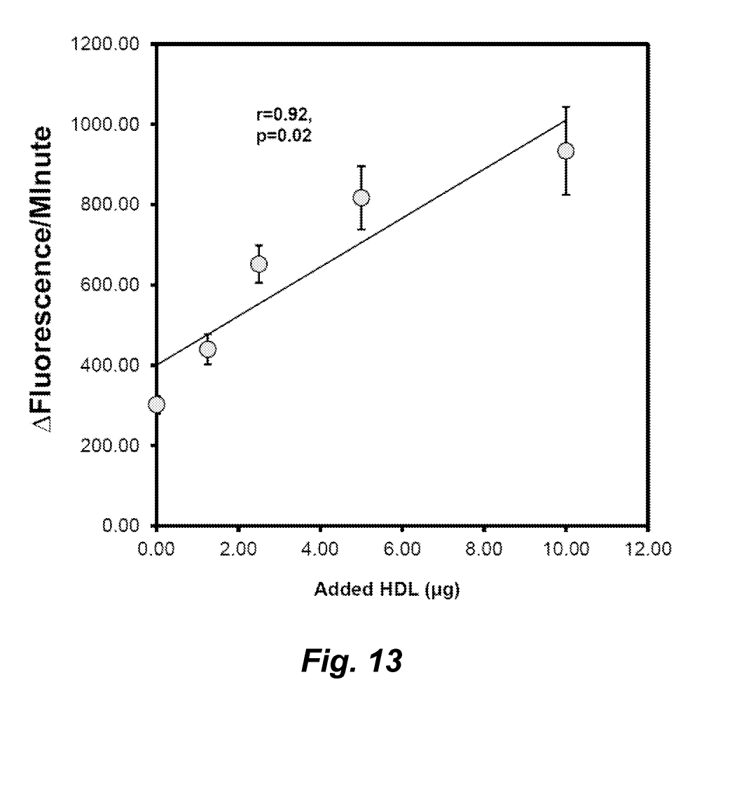

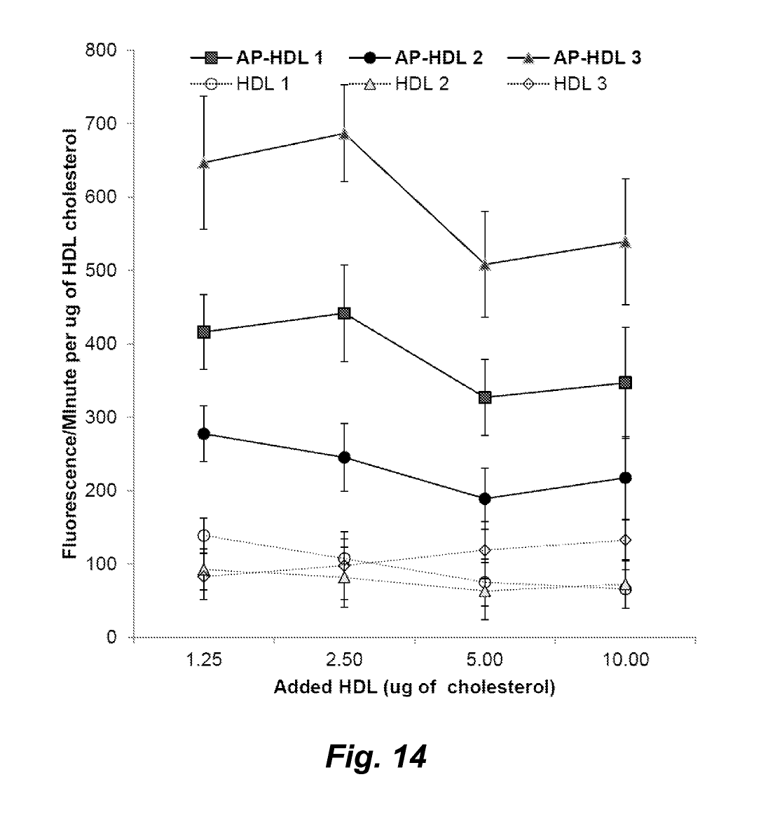

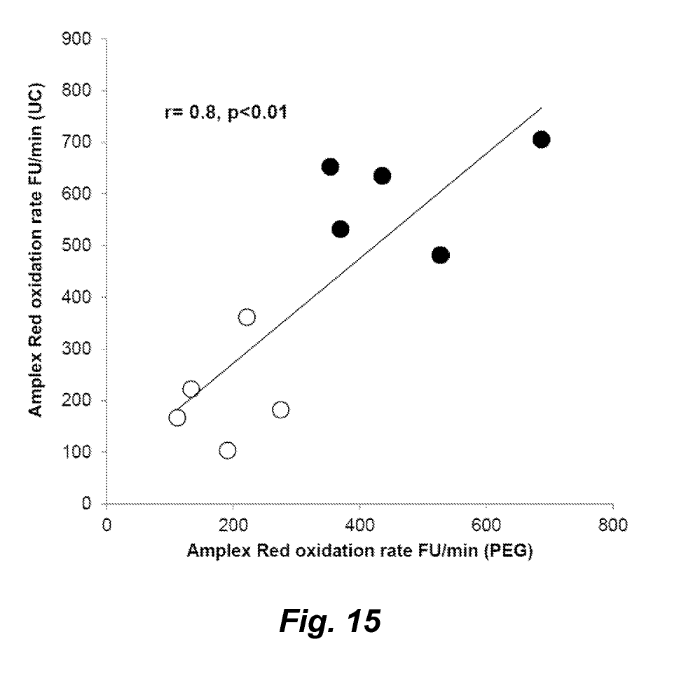

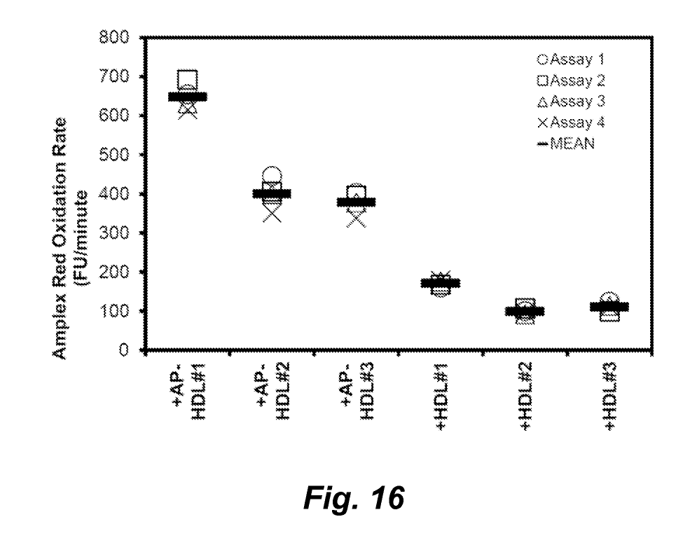

We used AMPLEX® RED in the presence of HRP to specifically quantify the rate of lipid peroxidation (HRA) of a specific amount of HDL cholesterol. The AMPLEX® RED assay could detect a concentration dependent increase in the amount of hydroperoxides associated with increasing amount of added HDL cholesterol (FIG. 13). In addition, the AMPLEX® RED assay could reliably quantify the content of hydroperoxides associated with a specific amount of HDL cholesterol when ≦10 μg of HDL is added (FIG. 14). Comparable results irrespective of the method of HDL isolation were obtained using the AMPLEX® RED assay to measure HRA among different samples (FIG. 15). Finally, the AMPLEX® RED could reliably measure HRA with low interassay and intra-assay experimental variability (<11%) irrespective of the used method of HDL isolation (FIG. 16).

The AMPLEX® RED Assay can Detect Dysfunctional HDL In Vivo.

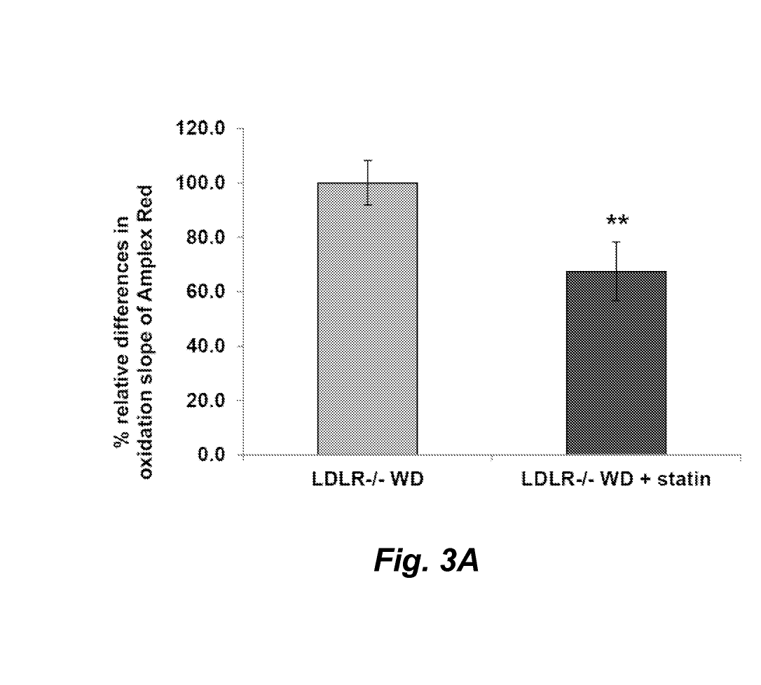

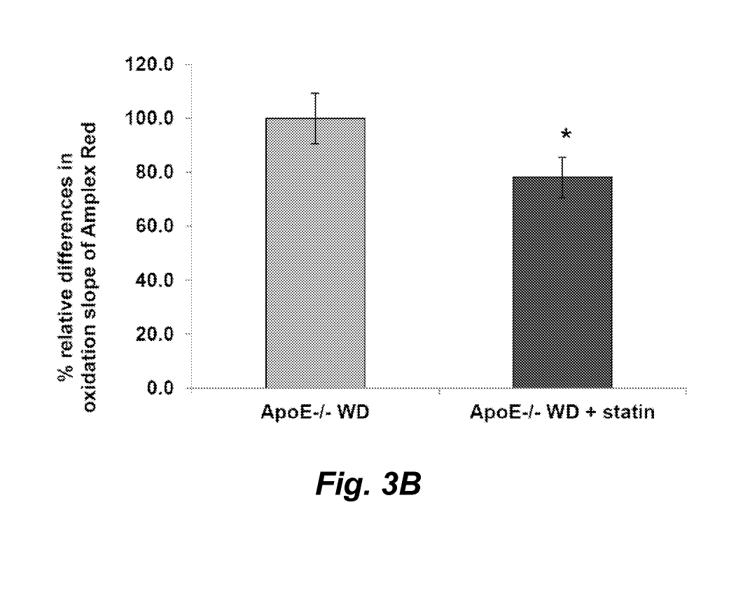

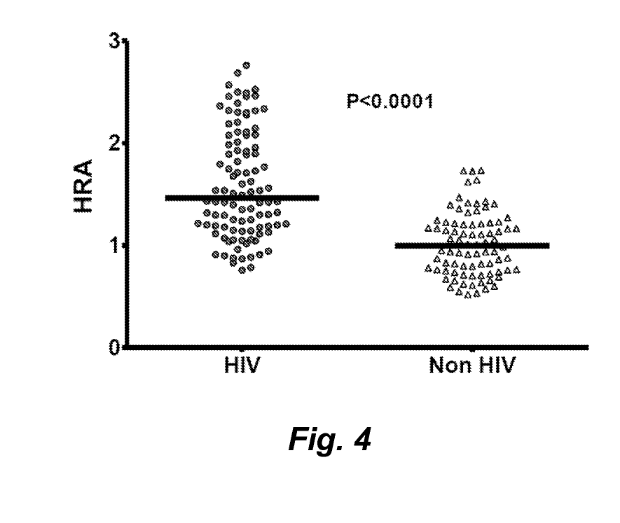

To further validate the new method we used the AMPLEX® RED assay to detect dysfunctional HDL in conditions in which dysfunctional HDL is known to be present such as established animal models of atherosclerosis (Watanabe et al. (2009) J. Biol. Chem. 284(27): 18292-18301; Kelesidis et al. (2011) J. Lipid. Res. 52(12): 2341-2351), and Human Immunodeficiency Virus infection (HIV) (Kelesidis et al. (2011) J. Lipid. Res. 52(12): 2341-2351). The AMPLEX® RED assay could detect established effect of statins on functional properties of HDL in animal models of atherosclerosis such as LDLR^{-/-} (FIG. 3A) and ApoE−/− mice (FIG. 3B). In addition, using samples from a previous cohort of HIV patients known to have dysfunctional HDL by using previously established assays of HDL function (Kelesidis et al. (2011) J. Lipid. Res. 52(12): 2341-2351; Kelesidis et al. (2012) Lipids Health Dis. 11: 87), the AMPLEX® RED assay confirmed that these patients had higher HRA compared to healthy controls (FIG. 4). Thus, using this methodology we demonstrate that HDL from patients with dysfunctional HDL has a higher rate of lipid peroxidation of a specific amount of HDL (HRA) compared to HDL from healthy patients.

Results from the AMPLEX® RED Assay Correlate with Previously Validated Cell-Based and Cell-Free Assays.

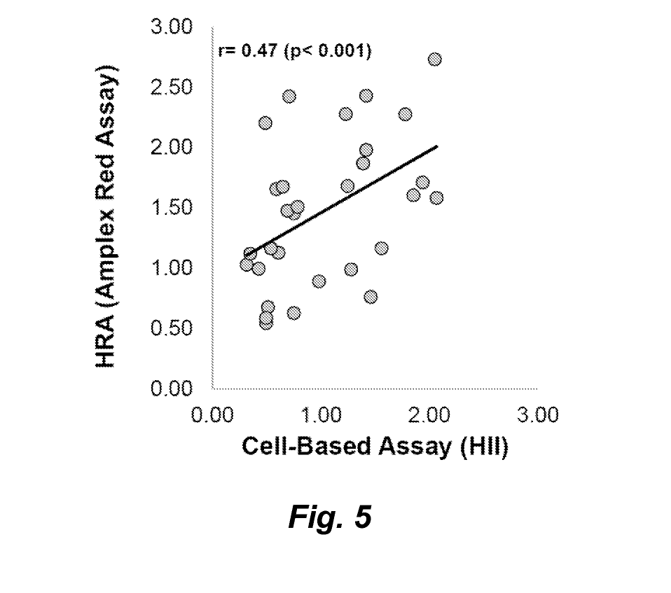

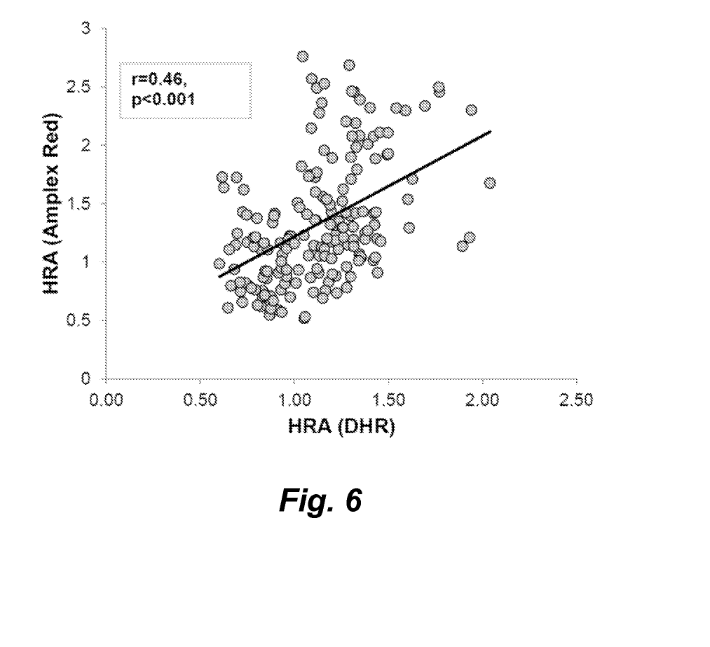

To compare the results of the AMPLEX® RED assay to those obtained using a validated cell-based assay (Watson et al. (2011) J. Lipid. Res. 52(2): 361-373; Kelesidis et al. (2011) J. Lipid. Res. 52(12): 2341-2351) 30 HDL samples were assessed using both assays (FIG. 5). Comparing HRA as measured with AMPLEX® RED to the HDL inflammatory index, there was a strong positive correlation (r=0.47, p<0.001). Moreover, to compare the results of the AMPLEX® RED assay to those obtained using a validated cell-free assay (Movva and Rader (2008) Clin. Chem. 54(5): 788-800) 60 HDL samples were assessed using both assays (FIG. 6). HRA as measured with AMPLEX® RED correlated significantly to the HRA as measured with DHR (r=0.46, p<0.001).

Standardization of the AMPLEX® RED Assay of HDL Function.

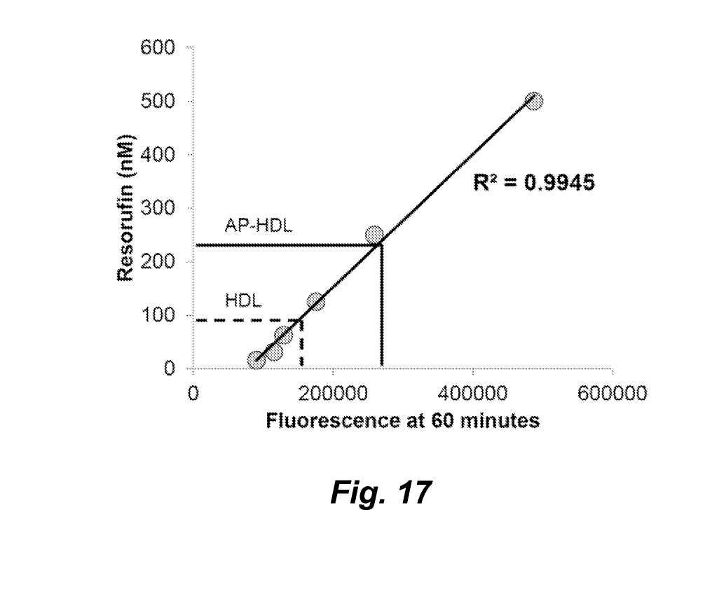

The lack of standardization of assays of HDL function may limit comparison of results between studies. An AMPLEX® RED stock solution of resorufin can be used to prepare a standard curve to determine the moles of product produced in the AMPLEX® RED reaction (Mishin et al. (2010) Free Radic. Biol. Med. 48(11): 1485-1491; Kalyanaraman et al. (2012) Free Radic. Biol. Med. 52(1): 1-6). Using this approach the fluorescence readout that corresponds to HRA can be expressed as the rate of formation of specific amount of resorfurin per specific unit of time (e.g., μM resorfurin/min) and this readout can be directly compared among different experiments especially since all the reagents used in the AMPLEX® RED assay, including all buffers, are commercially available and accessible to all researchers (FIG. 17).

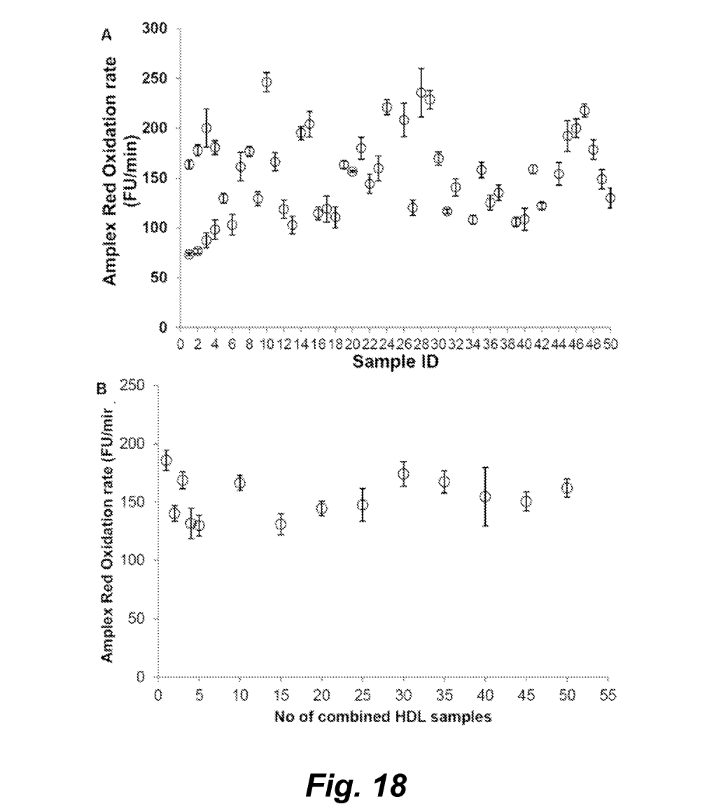

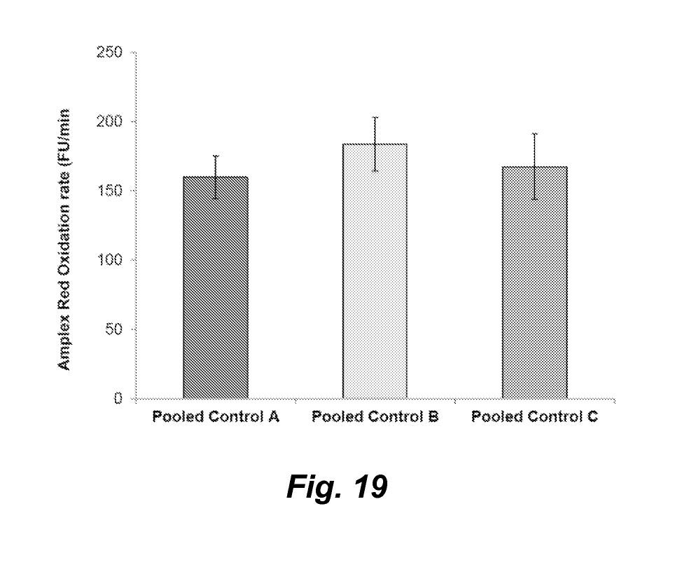

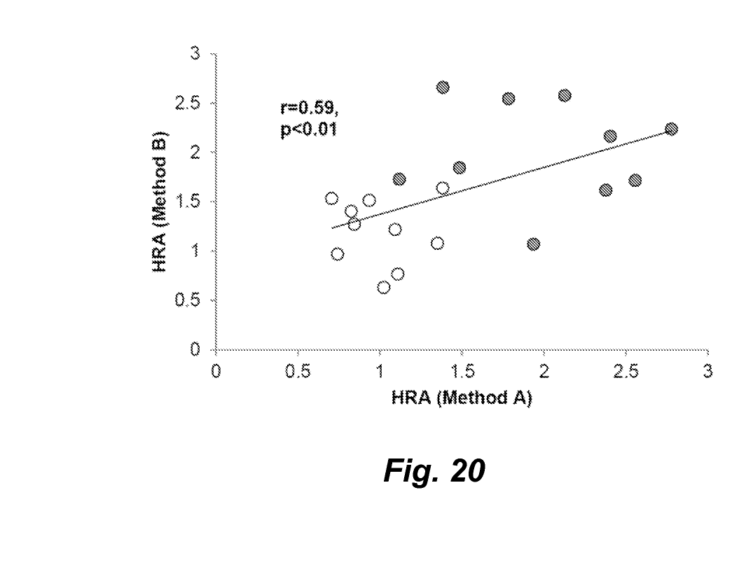

We also used a control sample which is prepared from pooled HDL samples isolated from plasma blood bank specimens to standardize the assay (similarly to INR, International Normalized Ratio) as previously (Kelesidis et al. (2013) Lipids Health Dis. 2013; 12: 23) (FIGS. 18-19). Since eligibility criteria for blood bank donors are well defined worldwide (Boulton (2008) Transfus. Med. 18(1): 13-27; Price (2008) Standards for blood banks and transfusion services. 25th ed, Bethesda (Md.), American Association of Blood Banks), this approach may be used to create a universal control even when pooled controls from different blood banks are used to compare results of assays of HDL function between different studies (FIG. 19). Finally the AMPLEX® RED assay can be further standardized by using the HDL concentration as determined by the clinical laboratory (a well-standardized measurement) rather than the HDL cholesterol concentration as determined by a cholesterol assay (as described in Methods) to adjust the fluorescence readout for the amount of HDL cholesterol in each sample (FIG. 20).

Freeze-Thaw can Affect HRA as Measured by the AMPLEX® RED Assay.

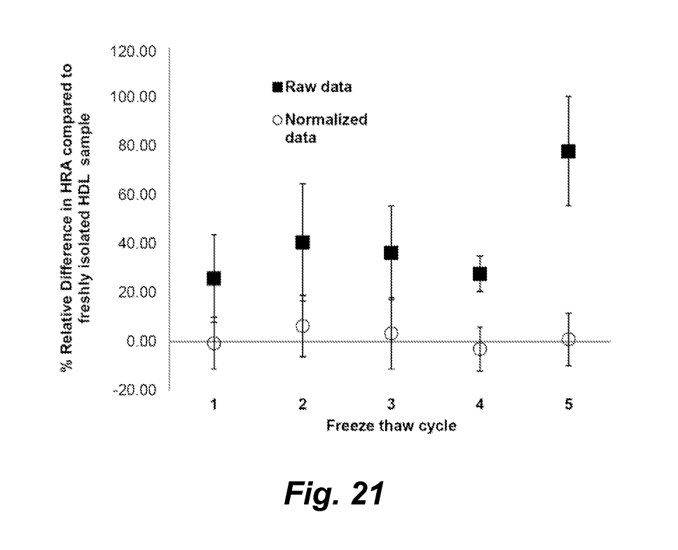

Similar to our previous results (Kelesidis et al. (2012) Lipids Health Dis. 11: 87; Kelesidis et al. (2011) J. Lipid. Res. 52(12): 2341-2351) we found that cryopreserved samples up to one freeze-thaw cycle can reliably be used to determine HRA in the AMPLEX® RED assay (Kelesidis et al. (2012) Lipids Health Dis. 11: 87) (unpublished data; FIG. 21). The standardization method with the pooled control may minimize the effect of multiple freeze-thaw cycles on determination of HRA using the AMPLEX® RED assay (FIG. 21).

Matrix Effects can Affect HRA as Measured by the AMPLEX® RED Assay.

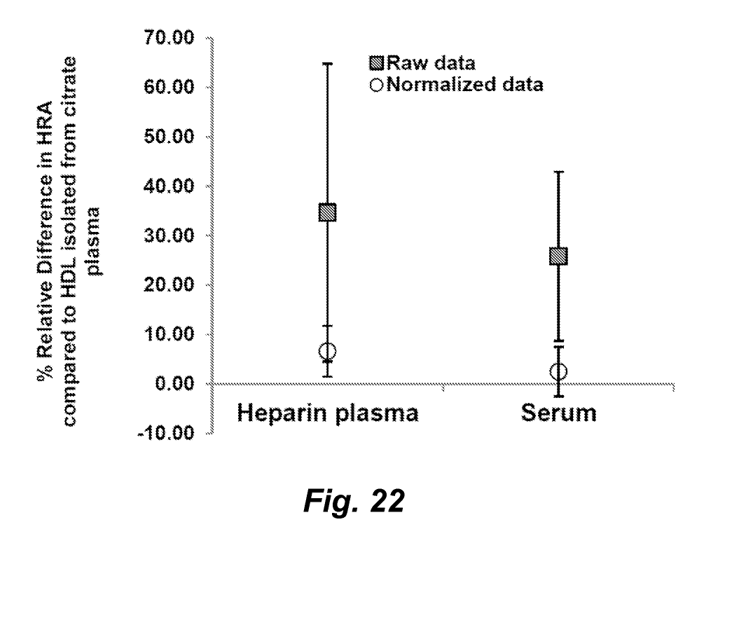

Similar to our previous methodology (Kelesidis et al. (2012) Lipids Health Dis. 11: 87; Kelesidis et al. (2011) J. Lipid. Res. 52(12): 2341-2351), we have confirmed the effects of different matrices such as plasma or serum on biochemical assays of HDL function and we conclude that plasma citrate or serum should be best used to determine HDL redox activity in the AMPLEX® RED assay since heparin and EDTA can affect determination of ROS in biological fluids (Wardman (2007) Free Radic. Biol. Med. 43(7): 995-1022) (unpublished data; FIG. 22). The standardization method with the pooled control tended to minimize the effect of different matrices on determination of HRA (FIG. 22).

Sample Handling and Long Term Cryopreservation can Affect HRA as Measured by the AMPLEX® RED Assay.

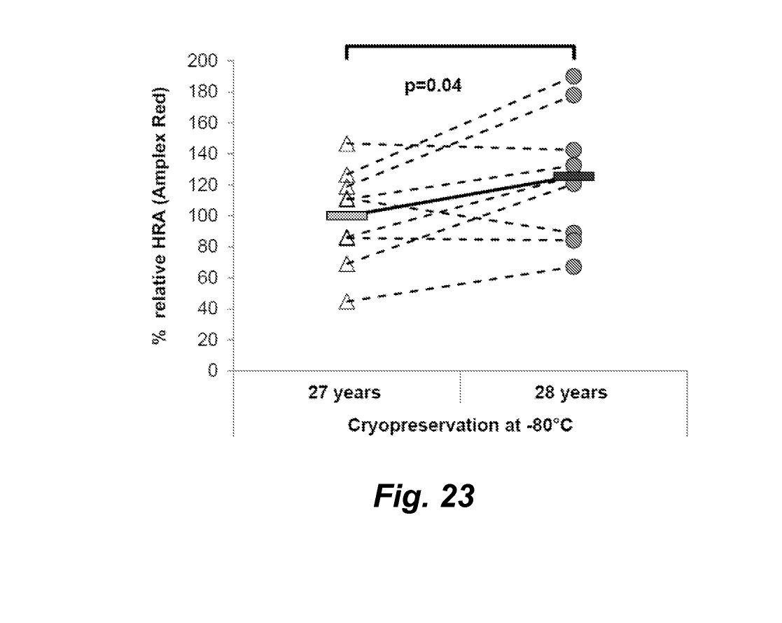

We then determined the effects of long-term storage of blood specimens at −80° C. on HRA. We found that long-term storage of blood samples did not significantly change the ability of the assay to demonstrate relative differences in the HRA between different groups of patients (FIG. 23). However, there was a modest increase in the HRA of a specific amount of HDL cholesterol of the same samples that had been stored an additional year. Thus, although artifactual oxidation of protein and/or lipid components of HDL and plasma/serum may affect assays that measure oxidative state of HDL to a certain extent, the relative differences in the oxidative state of HDL between different groups can still be quantified using cryopreserved samples. Ideally, freshly isolated (not cryopreserved) HDL should be used for biochemical assays of HDL function that measure HRA.

The Immunoaffinity Capture of HDL can be Used in the AMPLEX® RED Assay of HDL Function to Isolate HDL and Minimize Albumin Contamination.

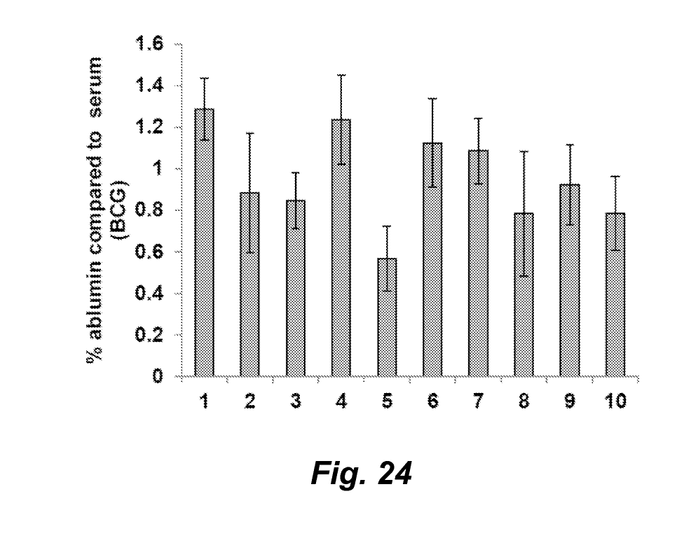

Although PEG precipitation is a reproducible, high throughput method to extract HDL from patient serum (Patel et al. (2011) J. Am. Coll. Cardiol. 58(20): 2068-2075), a major issue with this method is contamination of the HDL with other plasma proteins, especially albumin. Albumin represents a very abundant and important circulating antioxidant that binds ROS (Roche et al. (2008) FEBS Lett. 582(13): 1783-1787), removes lipid peroxidation products (Jayaraman et al. (2007) Biochemistry 46(19): 5790-5797) and can also significantly interfere with the fluorescent readout in biochemical assays of HDL function (Kelesidis et al. (2012) Lipids Health Dis. 11: 87). Although albumin contamination of HDL can be minimized using ultracentrifugation to isolate HDL this method is not high throughput and does not allow use of this assay in large scale studies. We have previously used immunoaffinity capture of HDL to study changes in the proteome associated with dysfunctional HDL (Watanabe et al. (2009) J. Biol. Chem. 284(27): 18292-18301; Watanabe et al. (2007) J. Biol. Chem. 282(32): 23698-23707; Watanabe et al. (2012) Arthritis Rheum. 64(6): 1828-1837). Commercially available total HDL ELISA kits can be used to capture HDL in 96-well plates when a specific volume of blood, purified HDL or apo-B depleted serum is added. For this purpose we have validated and used two commercially available kits (Kit A: Genway, San Diego, Calif.; Kit B: Biotang Inc, Waltham, Mass.). The immunogen is total human HDL derived from pooled plasma from healthy donors and the antibody is chicken (kit A) and mouse (kit B) anti-HDL. The sensitivity of detection of total HDL is 1.5 ng/ml for both kits. According to the manufacturer's instructions, by immunoelectrophoresis and ELISA the antibodies in these kits react specifically with human HDL and not with other human serum proteins including human IgG and human serum albumin (package insert, personal communication). Indeed, using immunoaffinity capture of HDL and 2 different methods to detect albumin content, we showed that the HDL captured on 96-well plates is largely free of albumin (FIG. 24).

AMPLEX® RED can Reliably Measure Lipid Peroxidation of a Specific Amount of HDL Isolated Using Immunoaffinity Capture of HDL.

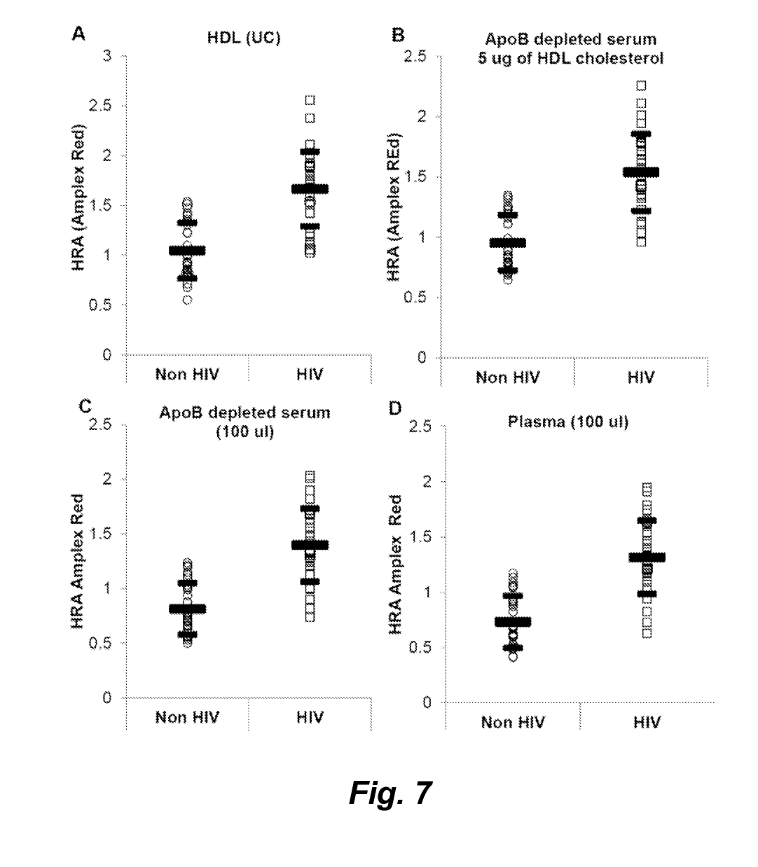

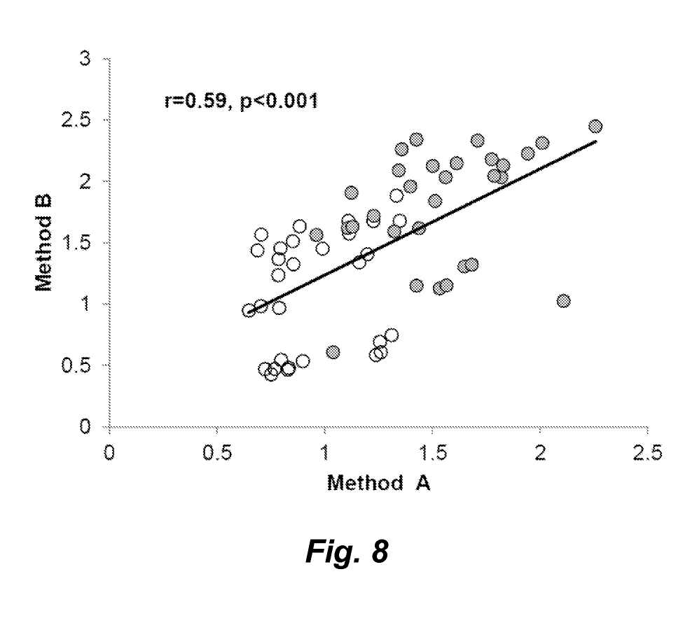

We used AMPLEX® RED in the presence of HRP and in combination with immunoaffinity capture of HDL (for HDL isolation) to specifically quantify the rate of lipid peroxidation (HRA) of a specific amount of HDL cholesterol. Using this methodology with different matrices we demonstrate that HDL from patients with dysfunctional HDL has a higher rate of lipid peroxidation of a specific amount of HDL compared to HDL from healthy patients (FIG. 7). However, since the proteome of dysfunctional HDL has not been fully elucidated (Watanabe et al. (2012) Arthritis Rheum. 64(6): 1828-1837), it is possible that dysfunctional HDL may bind less efficiently to different commercially available HDL antibodies and the total amount of HDL protein may not be directly compared between different subjects. Thus, to determine whether the approach of immunoaffinity capture of HDL depends on the quality of commercially available HDL antibodies, we used 2 different commercially available HDL kits with the AMPLEX® RED assay (kit A and B) to measure HRA in 60 samples. We found comparable results (FIG. 8) which indicate that the detected differences in HRA are real and not artificial secondary to variations in binding of HDL to the antibody. Thus, the suggested method can allow high throughput isolation of HDL and in situ detection of HRA that is associated with HDL function.

The Immunoaffinity Capture of HDL can be Used to Detect Total HDL Protein Concentration that can Also be Used to Normalize the Fluorescent Readout in the AMPLEX® RED Assay.

Using specific amounts of commercially available purified HDL (Sigma) (5 μg of HDL cholesterol), purified HDL isolated by ultracentrifugation (5 μg of HDL cholesterol), plasma (100 μl), or apo-B depleted serum (100 μl) it was confirmed that a specific amount of total HDL protein can be detected using the antibodies in these kits according to the manufacturer's instructions (data not shown). Alternatively the protein amount of the HDL captured in each well can be quantified by the BCA Protein Assay as previously described (Watanabe et al. (2012) Arthritis Rheum. 64(6): 1828-1837). However when the fluorescent readout (as determined in FIG. 7) was normalized by the protein HDL content in each well (determined by either the ELISA method or the BCA method) there were no significant differences in the normalized HRA between HDL from patients known to have dysfunctional HDL and healthy patients (data not shown).

The AMPLEX® RED-Based Assay of HRA in Combination with HDL Immunocapture Yields Reproducible Measurements.

To assess which commercially available HDL antibody used to capture HDL in 96-well plates gives the least experimental variability, 5 HDL samples were assessed in four independent experiments using 5 different commercially available kits (kits A-E as described in Methods). The intra-assay variability between the quadruplicates, ranged from 5.4 to 12.1%. Between independent replicates of the experiment, inter-assay variability for each of the samples ranged from 6.2 to 19.7%. From all 5 different commercially available kits (data not shown), kit B had the least experimental variability with an inter-assay variability ranging from 5.2 to 7.8% (mean 6.7%) and inter-assay variability ranging from 5.4 to 10.5% (mean 8.2%). Finally, from all the 4 different matrices used for HDL capture as outlined above, for a specific HDL antibody addition of a specific volume of plasma (100 μl) gave the least experimental variability (data not shown), followed by addition of a specific volume of apoB depleted serum (100 μl). Thus, using specific volume of plasma as input to 96 well plates for HDL isolation, kit B for HDL capture, and normalization of the fluorescent readout (that corresponds to HRA) by the HDL cholesterol concentration (as measured by the clinical lab) was the most reproducible method to measure HRA.

The HRA as Measured with the Novel Assay has the Potential to be Used as a Marker of Cardiovascular Disease in Humans.

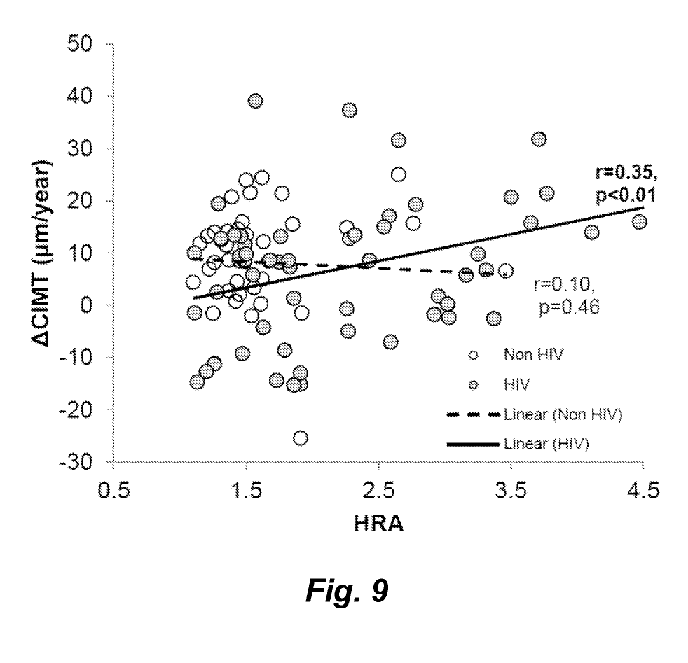

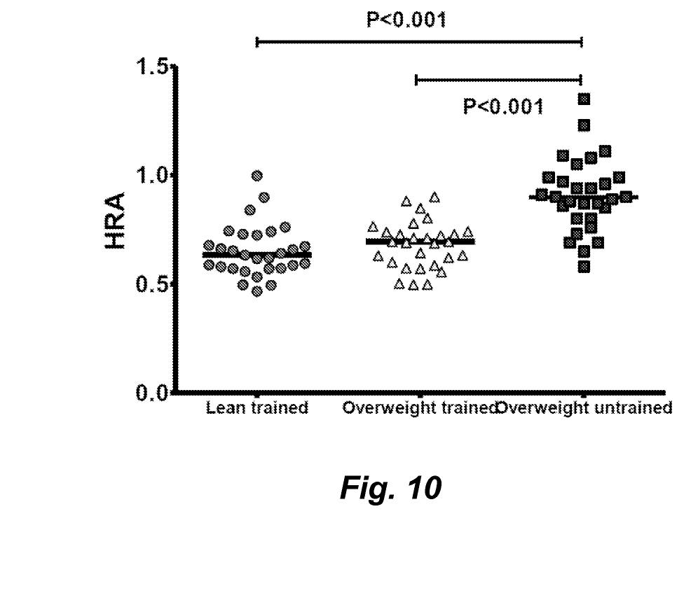

To validate the utility of the new assay to measure HRA as a marker of disease in humans, HRA was measured blindly using blood samples from a previously described cohort of 55 HIV infected subjects and 36 uninfected matched controls (Kelesidis et al. (2012) J. Infect. Dis. 206(10): 1558-1567) and the AMPLEX® RED assay. We found that HRA was independently associated with progression of subclinical atherosclerosis in HIV-infected subjects (FIG. 9).

The HRA as Measured with the Novel Assay can be Used as a Marker of Biologic Processes in Humans.

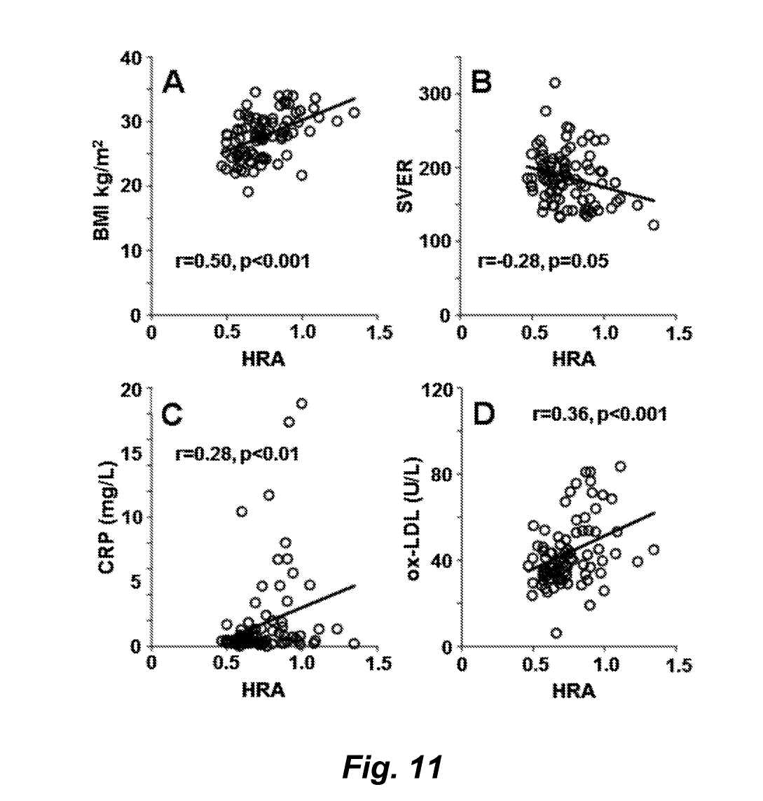

To further validate the utility of the new assay to measure HRA as a marker of HDL function and other physiological processes in humans, HRA was measured blindly in a previously established cohort of 90 subjects that looked into the effect of exercise on metabolic and other physiological parameters. Using the samples from this study and the AMPLEX® RED assay of HDL function we found that exercise improved HDL function, similarly to previous studies (FIG. 11) (Roberts et al. (2006) J. Appl. Physiol. 101(6): 1727-1732; Volkmann et al. (2010) Arthritis Care Res. 62(2): 258-265). Additionally, we assessed associations of HRA with indices of vascular and metabolic disease (FIG. 11). HRA (adjusted for HDL cholesterol concentration as outlined above) had significant negative associations with the subendocardial viability ratio (r=−0.21, p=0.05), relative strength (r=−0.45, p<0.001), homeostatic model assessment of insulin resistance (r=−0.25, p=0.02), HDL (r=−0.92, p=<0.00001), adiponectin (r=−0.29, p<0.01) and significant positive associations with anthropometric parameters of obesity such as BMI (r=0.50, p=<0.0001), waist circumference (r=0.59, p=<0.0001), trunk fat (r=0.56, p=<0.0001), total fat mass (r=0.55, p=<0.0001), C-reactive protein (CRP) (r=0.28, p=<0.001), fasting glucose (r=0.23, p=0.03), fasting insulin (r=0.21, p=0.04), amylin (r=0.23, p=0.04), leptin (r=0.48, p<0.001), oxidized LDL (r=0.36, p<0.001), triglycerides (r=0.36, p<0.001). These associations remained significant in multivariate analysis after adjusting for metabolic and anthropometric parameters (unpublished data). These pilot data demonstrate our ability to measure HRA and associate cardiovascular and metabolic risk phenotypes with these measures.

### DISCUSSION

Growing evidence suggests that HDL varies significantly in its phenotype and influence on cardiovascular disease risk (Navab et al. (2011) Nat. Rev. Cardiol. 8(4): 222-232; Navab et al. (2006) Nat. Clin. Pract. Endocrinol. Metab. 2(9): 504-511). HDL particles are heterogeneous in shape, density, size, composition and have multiple functional properties such as reverse cholesterol transport (RCT), anti-oxidant, anti-inflammatory, and antithrombotic activities (Navab et al. (2011) Nat. Rev. Cardiol. 8(4): 222-232). HDL are “Janus-like” lipoproteins with the capacity to be anti-inflammatory in the basal state and proinflammatory during acute-phase responses (Navab et al. (2011) Nat. Rev. Cardiol. 8(4): 222-232; Navab et al. (2009) J. Lipid. Res. 2009; 50 Suppl: S145-S149). Previous work has also suggested dysfunctional HDL to be pronounced in chronic inflammatory conditions that predispose to atherosclerosis (Navab et al. (2009) J. Lipid. Res. 2009; 50 Suppl: S145-S149; Navab et al. (2004) J. Lipid. Res. 45(6): 993-1007; Navab et al. (2006) Nat. Clin. Pract. Endocrinol. Metab. 2(9): 504-511; Navab et al. (1996) Arterioscler. Thromb. Vasc. Biol. 16(7): 831-842). This impaired (dysfunctional) HDL, is characterized by (i) decreased levels and activity of anti-inflammatory, antioxidant factors (Navab et al. (2011) Nat. Rev. Cardiol. 8(4): 222-232; Navab et al. (2009) J. Lipid. Res. 2009; 50 Suppl: S145-S149); (ii) gain of pro-inflammatory proteins (Mackness et al. (2004) Am. J. Cardiovasc. Drugs, 4(4): 211-217); (iii) increased lipid hydroperoxide (LOOH) content (Van Lenten et al. (1995) J. Clin. Invest. 96(6): 2758-2767); (iv) reduced potential to efflux cholesterol (Navab et al. (2001) Arterioscler. Thromb. Vasc. Biol. 21(4): 481-488); and (v) diminished ability to prevent LDL oxidation (Hayek et al. (1994) Biochem. Biophys. Res. Commun. 205(2): 1072-1078). Measuring the functional status of HDL, rather than HDL cholesterol concentration, may be more informative in predicting cardiovascular disease risk (Navab et al. (2005) Ann. Med. 37(3): 173-178). Unfortunately, the most widely used assays for HDL function have been cell-based assays (Navab et al. (2011) Nat. Rev. Cardiol. 8(4): 222-232) which are highly labor-intensive and subject to substantial assay variation (Patel et al. (2009) J. Am. Coll. Cardiol. 53(11): 962-971; Undurti et al. (2009) J. Biol. Chem. 284(45): 30825-30835; Van Lenten et al. (2007) J. Lipid. Res. 48(11): 2344-2353) since the optimal method for performing such studies with regard to donor cells, type of acceptor and type of readout has yet to be determined. Thus, cell based assays of HDL function are technically prohibitive, especially for use in large-scale clinical studies.

Cell-free assays of HDL function may be more precise than cell-based assays because they measure a biochemical rather than biologic phenomenon. Thus, we previously developed a cell free assay based on the fluorochrome DCF that assessed the HDL inflammatory index (HII), a measure of the capacity of HDL to prevent the formation or to inactivate oxidized phospholipids produced by LDL (Movva and Rader (2008) Clin. Chem. 54(5): 788-800). This was based on our demonstration that levels of ROS (such as lipid hydroperoxides produced from oxidation of lipoproteins) are significantly associated with inflammatory properties of HDL that are measured by the HII (Navab et al. (2001) J. Lipid. Res. 42(8): 1308-1317). This assay has been previously used in multiple studies in humans (Id.) and this measurement of HDL has been associated with other measures of HDL function such protein biomarkers associated with dysfunctional HDL (Khera et al. (2011) N. Engl. J. Med. 364(2): 127-135; Patel et al. (2011) J. Am. Coll. Cardiol. 58(20): 2068-2075; Watanabe et al. (2009) J. Biol. Chem. 284(27): 18292-18301; Morgantini et al. (2011) Diabetes, 60(10): 2617-2623; Imaizumi et al. (2010) Drug Metab. Lett. 4(3): 139-148; Charles-Schoeman et al. (2009) Arthritis Rheum. 60(10): 2870-2879; Watanabe et al. (2007) J. Biol. Chem. 282(32): 23698-23707; Watanabe et al. (2012) Arthritis Rheum. 64(6): 1828-1837), HDL anti-inflammatory function measured as the ability of test HDLs to inhibit LDL-induced monocyte chemotactic activity in human aortic endothelial cell monolayers (Watanabe et al. (2009) J. Biol. Chem. 284(27): 18292-18301; Charles-Schoeman et al. (2009) Arthritis Rheum. 60(10): 2870-2879; Watanabe et al. (2012) Arthritis Rheum. 64(6): 1828-1837) and measurement of oxidized fatty acids in HDLs (Morgantini et al. (2011) Diabetes, 60(10): 2617-2623; Charles-Schoeman et al. (2009) Arthritis Rheum. 60(10): 2870-2879). It has also been validated in vivo in animal models of atherosclerosis (Morgantini et al. (2011) Diabetes, 60(10): 2617-2623). Furthermore, HDL function measured using this assay has been correlated with systemic inflammation in coronary heart disease (CHD) patients (Imaizumi et al. (2010) Drug Metab. Lett. 4(3): 139-148; Watanabe et al. (2007) J. Biol. Chem. 282(32): 23698-23707) and cardiovascular disease outcome (Patel et al. (2011) J. Am. Coll. Cardiol. 58(20): 2068-2075). However, the oxidative instability of DCF-DA, variations in the donor LDL used in the assay and increased lipid-probe interactions of two lipoproteins used in the same biochemical reaction increased experimental variability of this method (Watanabe et al. (2007) J. Biol. Chem. 282(32): 23698-23707).

Recent interest has focused on the functional consequences of HDL oxidation. Oxidation could conceivably contribute to the formation of dysfunctional HDL, proposed to be present in humans with cardiovascular disease (Kelesidis et al. (2011) J. Lipid. Res. 52(12): 2341-2351). One potentially important pathway for generating dysfunctional HDL via oxidation involves myeloperoxidase that mediates conversion of protein tyrosine residues to 3-chlorotyrosine, and methionine residues to methionine sulfoxide (MetO) (Barter et al. (2004) Circ. Res. 95(8): 764-772). In addition, MetO can also be formed from exposure of HDL's major protein, apolipoprotein A-I (apoA-I) to H₂O₂ (Daugherty et al. (1994) J. Clin. Invest. 94(1): 437-444) or lipid hydroperoxide (Anantharamaiah et al. (1988) J. Lipid. Res. 29(3): 309-318), the latter generated during the oxidation of HDL lipids. Reactive oxygen species (ROS) such as 1e-oxidants (i.e., hydroxyl radical and metal ions) have previously been shown to oxidize tyrosine and methionine residues (Garner et al. (1998) J. Biol. Chem. 273(11): 6080-6087; Garner et al. (1998) J. Biol. Chem. 273(11): 6088-6095) which can have dramatic consequences on the functions of apoA-I/HDL, including reverse cholesterol transport (Wang et al. (2009) J. Lipid. Res. 50(3): 586-594).

In view of the significance of HDL redox activity for HDL function and the previous demonstration that the oxidation rate of DHR can be used to quantify redox activity in extracellular matrix such as plasma (Oram and Heinecke (2005) Physiol. Rev. 85(4): 1343-1372), we developed a biochemical assay of HDL function that is based on DHR and in which the endogenous HRA is assessed in terms of the capacity of a specific amount of HDL cholesterol to engage in vitro redox cycling (Esposito et al. (2003) Blood 102(7): 2670-2677).

In contrast to DCFH which is unstable and prone to auto-oxidation, DHR is relatively stable, requires no activation and oxidizes at a predictable rate when exposed to room air (Kelesidis et al. (2011) J. Lipid. Res. 52(12): 2341-2351). In addition the kinetic approach for measuring oxidation rate during a linear phase, lends greater precision compared to a single endpoint measurement. This assay was validated in vitro (Id.) against an established monocyte chemotaxis assay of HDL function and in vivo (Id.).

High density lipoprotein is the major carrier of lipid hydroperoxides in human blood plasma (Amundson et al. (1999) J. Biochem. Biophys. Meth. 38(1): 43-52; Mishin et al. (2010) Free Radic. Biol. Med. 48(11): 1485-1491) and dysfunctional HDL had increased endogenous “ROS load” and redox activity (Bowry et al. (1992) Proc. Natl. Acad. Sci. USA, 89(21): 10316-10320). 1e-oxidants such as Off preferentially react with the lipoprotein's lipids and this causes lipid peroxidation with the resulting accumulation of hydroperoxides of phospholipids and cholesteryl-esters (Navab et al. (2011) Nat. Rev. Cardiol. 8(4): 222-232; Navab et al. (2001) J. Lipid. Res. 42(8): 1308-1317; Garner et al. (1998) J. Biol. Chem. 273(11): 6080-6087; Garner et al. (1998) J. Biol. Chem. 273(11): 6088-6095; Bowry et al. (1992) Proc. Natl. Acad. Sci. USA, 89(21): 10316-10320; Sattler et al. (1995) Free Radic. Biol. Med. 18(3): 421-429) that then oxidize apoA-I's methionine to MetO (60). We have previously shown that increased lipid hydroperoxide (LOOH) content is associated with dysfunctional HDL (Navab et al. (2001) Arterioscler. Thromb. Vasc. Biol. 21(4): 481-488; Castellani et al. (1997) J. Clin. Invest. 100(2): 464-474) and that oxidized HDL is dysfunctional HDL (Garner et al. (1998) J. Biol. Chem. 273(11): 6080-6087; Garner et al. (1998) J. Biol. Chem. 273(11): 6088-6095; Sattler et al. (1995) Free Radic. Biol. Med. 18(3): 421-429). However, although DHR may measure the capacity of HDL cholesterol to engage in vitro redox cycling (Kelesidis et al. (2011) J. Lipid. Res. 52(12): 2341-2351; Kelesidis et al. (2012) Lipids Health Dis. 11: 87), DHR is not a substrate for oxidation by H₂O₂ (Kelesidis et al. (2011) J. Lipid. Res. 52(12): 2341-2351) suggesting that lipid hydroperoxides in HDL are not promoting DHR oxidation. Thus the biochemical mechanism of the DHR assay of HDL function remains to be determined.

In addition, when apoB depleted serum and cryopreserved samples were used in the setting of larger studies, it was found that lipid-probe interactions that depend on the type and concentration of the lipid and the fluorescent probe, method of HDL isolation, freeze-thaw, different diluents and matrices and changes in pH may complicate interpretation of the results (Royall et al. (1993) Arch. Biochem. Biophys. 302(2): 348-355). The limitations of the DHR assay have been previously described in detail (Kelesidis et al. (2011) J. Lipid. Res. 52(12): 2341-2351; Kelesidis et al. (2012) Lipids Health Dis. 11:87). Thus, only fresh apoB depleted serum samples and purified HDL isolated by ultracentrifugation may be used with the DHR assay and this may limit its utility for large scale clinical studies.

Herein an approach is described to quantify HDL redox activity, a measure of HDL function, in a novel cell-free, biochemical assay. Fluorescent probe(s) were identified that meet the following criteria i) reliably and specifically quantify the rate of lipid peroxidation of a specific amount of HDL cholesterol; ii) enzymatic amplification of the measurement of ROS with would overcome lipid-probe-ROS interactions that may be a limitation of fluorescent biochemical assays of HDL function (Kelesidis et al. (2011) J. Lipid. Res. 52(12): 2341-2351; Kelesidis et al. (2012) Lipids Health Dis. 11: 87); iii) would measure ROS accurately despite the known challenges and limitations that most widely used fluorescent probes have for detecting and measuring ROS (Kelesidis et al. (2012) Lipids Health Dis. 11: 87).

AMPLEX® RED is a fluorogenic substrate with very low background fluorescence, that reacts with H₂O₂ with a 1:1 stoichiometry to produce highly fluorescent resorufin (Kagramanov and Lyman (2001) J. Amer. Med. Assoc. 285(7): 881). AMPLEX® RED can be oxidized by HRP which vastly increases the yield of resorufin (Mishin et al. (2010) Free Radic. Biol. Med. 48(11): 1485-1491; Kalyanaraman et al. (2012) Free Radic. Biol. Med. 52(1): 1-6) and this assay is a reliable method to continuously measure the extracellular formation of H₂O₂ (Id.) in 96 well plate format (Id.). We have previously used the AMPLEX® RED reagent to quantify ROS produced by oxidized lipids in cell culture supernatants (Amundson et al. (1999) J. Biochem. Biophys. Meth. 38(1): 43-52; Mishin et al. (2010) Free Radic. Biol. Med. 48(11): 1485-1491).

AMPLEX® RED in the presence of the enzyme cholesterol oxidase has been reliably used to quantify cholesterol content of HDL based on lipid peroxidation of HDL (DeMaio et al. (2006) Am. J. Physiol. Heart. Circ. Physiol. 2006; 290(2): H674-H683). Using a modification of this well described assay (FIG. 1), as shown herein, it was demonstrated that AMPLEX® RED, in the absence of cholesterol oxidase and for the same amount of HDL cholesterol, can detect differences in the rate of lipid peroxidation between different HDL samples that correspond to differences in HDL function.

The products of redox cycling are detected as time-dependent oxidation of the fluorogenic probe AMPLEX® RED that in the presence of HRP specifically quantifies the rate of lipid peroxidation of a specific amount of HDL cholesterol and the rate of reaction of the OH⁻ with AMPLEX® RED. In the presence of peroxidase, the AMPLEX® RED reagent reacts with H₂O₂ in a 1:1 stoichiometry to produce the red-fluorescent oxidation product, resorufin and this reaction has been used to detect as little as 10 picomoles of H₂O₂ in a 100 μL volume (50 nM) or 1×10⁻⁵ U/mL of HRP (Gutheil et al. (2000) Anal. Biochem. 287(2): 196-202; Peus et al. (1999) J. Invest. Dermatol. 112(5): 751-756; Richer and Ford (2001) Mol. Hum. Reprod. 7(3): 237-244). We demonstrate that this approach in combination with immunoaffinity capture of HDL for HDL isolation may overcome many confounding factors that affected the readout in the previously described cell free assays of HDL function and provides a measurement of HRA that correlates well with previously validated cell-based and cell-free assays of HDL function. Because a large proportion of cholesterol in blood is in the form of cholesteryl esters, cholesterol esterase is used to produce free cholesterol from cholesterol esters (Amundson et al. (1999) J. Biochem. Biophys. Meth. 38(1): 43-52; Mishin et al. (2010) Free Radic. Biol. Med. 48(11): 1485-1491). In addition the assay may be modified and cholesterol esterase may or may not be added in the AMPLEX® RED reagent so that peroxidation of HDL cholesterol in the form of cholesteryl esters versus free cholesterol can be determined (Id.).

This AMPLEX® RED-based cell-free assay improves upon the prior DHR-based cell-free assay. While also measuring the HRA the biochemistry of the AMPLEX® RED fluorochrome and its ability to detect ROS and lipid hydroperoxides is well established. In the absence of cholesterol oxidase the AMPLEX® RED detects the intrinsic hydroperoxide content of a specific amount of HDL cholesterol. Moreover, the use of immunoaffinity capture may allow HDL isolation and use of this method in large scale studies and removal of much of the albumin bound to the HDL particle that may alter the association of ROS with lipoproteins (Kelesidis et al. (2011) J. Lipid. Res. 52(12): 2341-2351). Finally, the inter-assay variability of <15% compares favorably with cell-based assays of HDL function, which have variability of >15% (Roche et al. (2008) FEBS Lett. 582(13): 1783-1787).

In addition using animal models of atherosclerosis and two different human studies it was shown that this assay can be used as a marker of cardiovascular disease and biologic processes in vivo. The correlation of HRA as measured by the AMPLEX® RED assay to the biologic readout of HDL in a cell-based assay is consistent with a proposed mechanism whereby HDL exerts its effects through modulating ROS (Navab et al. (2004) J. Lipid. Res. 45(6): 993-1007; Navab et al. (2006) Nat. Clin. Pract. Endocrinol. Metab. 2(9): 504-511), and suggests that the assay accurately reflects HDL function. Further support for the biological relevance of this measurement is the finding that for the same amount of HDL cholesterol the HRA was significantly reduced in HDL from statin treated mice with atherosclerosis compared to HDL from non-statin treated mice. Indeed, treatment of these mice with statins has previously been shown to reduce inflammatory properties of HDL (Navab et al. (2001) J. Lipid. Res. 42(8): 1308-1317) and was demonstrated herein that the AMPLEX® RED assay can detect the favorable effect of statins on functional properties of HDL. Moreover, the AMPLEX® RED assay can detect dysfunctional HDL in patients with HIV infection confirming our previous results (Navab et al. (2005) Arterioscler. Thromb. Vasc. Biol. 25(7): 1426-1432). The fact that the assay quantifies HRA and that the HRA levels correlate with surrogate measures of cardiovascular disease and other physiological parameters in humans such as obesity and insulin resistance increases its applicability to biological samples, at least in the context of cardiovascular diseases. Without being bound by a particular theory, it is believed this is because 1e-oxidants (i.e., hydroxyl radical) contribute to oxidative modifications taking place in the affected arterial wall during atherogenesis (Kelesidis et al. (2011) J. Lipid. Res. 52(12): 2341-2351; Kelesidis et al. (2012) Lipids Health Dis. 11: 87; Kelesidis et al. (2011) Lipids Health Dis. 10: 35). Thus, while “oxidized HDL” is not a chemically defined term, the oxidation rate of AMPLEX® RED corresponds to the intrinsic HRA of specific amount of HDL lipoproteins.

Finally, it was demonstrated that matrix effects, freeze thaw, sample handling and long term storage of blood specimens at −8° C. can affect the HRA but the relative differences in HRA between different samples can still be reliably detected with the AMPLEX® RED reagent, in either serum or non EDTA plasma, fresh or cryopreserved samples, and with up to 2 freeze-thaw cycles. However, the oxidative modifications occurring to HDL in the diseased artery wall are conceivably more complex (Stocker and Keaney (2004) Physiol. Rev. 84(4): 1381-1478) and HDL is subject to continuous remodeling in vivo. This includes dissociation of apoA-I from the lipoprotein particle, a process that could be increased by oxidation (Id.).

In conclusion, this new assay offers a rapid method for measuring the redox properties of HDL. It yields results that correlate well with previously validated cell-based and cell-free assays of HDL function and can be used as a marker of cardiovascular disease and biologic processes in humans. This new technical approach offers a convenient tool for studies of the role of HDL functional phenotype in the development of atherosclerosis in vivo.

### Acknowledgements

Funding: This work was supported by RO1 grants HL095132 (JSC) and HL082823 (STR), and UCLA AIDS Institute and the UCLA Center for AIDS Research (AI28697). Partial funding for laboratory work was provided by the University of Washington's CVD and Metabolic Complications of HIV/AIDS Data Coordinating Center (5R01HL095126). The exercise work was supported by the American Heart Association (BGIA 0765139Y to C.K.R.), the National Heart, Lung and Blood Institute (P50 HL105188 to C.K.R.), and the National Institute of Diabetes and Digestive and Kidney Diseases (DK090406 to C.K.R.).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of evaluating HDL function, said method comprising:
contacting a sample comprising HDL with 10-acetyl-3,7-dihydroxyphenoxazine (AMPLEX® RED) or with AMPLEX® ULTRARED in the presence of horse radish peroxidase (HRP) in a reaction mixture to provide a measure of the endogenous hydroperoxide content of said HDL, wherein said hydroperoxide content is a measure of HDL redox activity (HRA) for the HDL in said sample and where elevated HRA is an indicator of dysfunctional HDL.

2. The method of claim 1, wherein said sample is contacted with 10-acetyl-3,7-dihydroxyphenoxazine (AMPLEX® RED).

3. The method of claim 1, wherein said sample is contacted with AMPLEX® ULTRARED.

4. The method according to any one of claims of claims 1-3, wherein said reaction mixture does not contain cholesterol oxidase.

5. The method according to any one of claims 1-4, wherein cholesterol esterase is added to the reaction mixture so that peroxidation of HDL cholesterol in the form of cholesteryl esters versus free cholesterol can be determined.

6. The method according to any one of claims of claims 1-5, wherein said detecting comprises detecting the fluorescence or change in florescence of said reaction mixture over a time interval of at least 5 minutes, or at least 10 minutes, or at least 15 minutes, or at least 30 minutes, or at least 45 minutes, or at least 1 hour.

7. The method of claim 6, wherein said fluorescence is quantified relative to the maximum fluorescence observed over the entire time interval.

8. The method according to any one of claims 6-7, wherein said fluorescence is quantified relative to the corresponding time point of a reference control sample.

9. The method according to any one of claims 6-8, wherein said detecting comprises determining the concentration of the oxidation product of said AMPLEX® RED® from a standard curve for the oxidation product at the same timepoint.

10. The method of claim 9, said determining the concentration from a standard curve is performed using regression analysis.

11. The method according to any one of claims 1-10, wherein said detecting comprises determining the mean fluorescence readout (slope) for said reaction mixture and normalizing the value by the HDL concentration of said sample.

12. The method according to any one of claims 1-11, wherein said sample is, or is derived from, non EDTA plasma.

13. The method according to any one of claims 1-11, wherein said sample is, or is derived from, serum.

14. The method of claim 13, wherein said sample is, or is derived from, apoB depleted serum.

15. The method according to any one of claims 1-14, wherein said sample is, or is derived from a fresh (unfrozen) sample.

16. The method according to any one of claims 1-14, wherein said sample is, or is derived from a cyropreserved sample.

17. The method according to any one of claims 1-16, wherein said sample comprises isolated HDL.

18. The method of claim 13, wherein said sample comprises HDL isolated by a method selected from the group consisting of ultracentrifugation, PEG precipitation, heparin MnCl₂ precipitation, sodium phosphotungstate precipitation, dextran sulfate precipitation, and immunoaffinity capture.

19. The method of claim 13, wherein said sample comprises HDL isolated by PEG precipitation.

20. The method of claim 13, wherein said sample comprises HDL isolated by immunoaffinity capture.

21. The method according to any one of claims 1-20, wherein said detecting comprises comparing, or normalizing, said measurement to a control.

22. The method of claim 21, wherein said control comprises a sample (e.g., a pooled sample) from healthy subjects.

23. The method according to any one of claims 21-22, wherein said control comprises a positive control.

24. The method of claim 23, wherein said positive control comprises a hydrogen peroxide (H₂O₂) working solution.

25. The method according to any one of claims 21-24, wherein said detecting comprises determining the difference between said measurement and the same measurement made for a negative control.

26. The method of claim 25, wherein said negative control comprises a reaction mixture without cholesterol.

27. The method according to any one of claims 1-26, wherein said detecting comprises determining the production of hydroxyradicals as a result of air oxidation of buffer based on the readout of a blank well that contains AMPLEX® RED® and subtracting the value from the fluorescent readout of test samples.

28. The method according to any one of claims 1-27, wherein said method is performed in a high throughput format.

29. The method according to any one of claims 1-28, wherein said method is performed in a multi-well plate.

30. The method according to any one of claims 1-28, wherein said method is performed in a microfluidic device.

31. The method of claim 30, wherein said method is performed in a droplet-based (segmented flow) microfluidic system.

32. The method according to any one of claims 1-31, wherein elevated HRA is an HRA greater than the HRA measured for HDL from a normal healthy subject of the same age and gender.

33. The method according to any one of claims 1-31, wherein elevated HR is an HRA greater than the HRA associated with an inflammatory index greater than 1.

34. A method of determining the presence or risk of atherosclerosis in a subject, said method comprising:
determining HDL redox activity (HRA) for HDL in a sample from said subject according to the method of any one of claims 1-33, wherein an elevated HRA as compared to that for a normal healthy subject indicates that said subject has or is at risk for atherosclerosis.

35. The method of claim 34, wherein the elevated HRA and/or a diagnosis based, at least in part, on said level is recorded in a patient medical record.

36. The method of claim 35, wherein said patient medical record is maintained by a laboratory, physician's office, a hospital, a health maintenance organization, an insurance company, or a personal medical record website.

37. The method according to any one of claims 34-36, wherein a diagnosis, based at least in part on the HRA level is recorded on or in a medic alert article selected from a card, worn article, or radiofrequency identification (RFID) tag.

38. The method according to any one of claims 34-37, wherein said HRA levels and/or a diagnosis based upon the HRA levels is recorded on a non-transient computer readable medium.

39. The method according to any one of claims 34-38, wherein the HRA level is determined as part of a differential diagnosis.

40. The method according to any one of claims 34-39, wherein said subject is a non-human mammal.

41. The method according to any one of claims 34-39, wherein said subject is a human.

42. A method for the treatment or prophylaxis of atherosclerosis, said method comprising:
identifying a subject that has an elevated HDL redox activity as compared to a normal healthy individual or population or as compared to the same individual at an earlier time, where said elevated HDL redox activity is determined by the method of any one of claims 1-33; and
performing further testing and/or treating said subject as a subject having or at elevated risk for atherosclerosis.

43. The method of claim 42, wherein said subject is prescribed an additional test and/or said additional tests are performed.

44. The method of claim 43, wherein said additional tests comprise one or more tests selected from the group consisting of blood tests for heart tissue damage or high risk for heart attack, electrocardiogram, stress test, coronary MRI, and coronary angiography.

45. The method of claim 44, wherein said additional test comprises a blood test selected from the group consisting of troponin I, T-00745, creatine phosphokinase (CPK), LDL, AST, ALT, and myoglobin.

46. The method of claim 44, wherein additional test comprise a stress test selected from the group consisting of an exercise tolerance test, a nuclear stress test, cardiac MRI stress, and a stress echocardiogram.

47. The method according to any one of claims 42-46, wherein said subject is prescribed a treatment and/or treated.

48. The method of claim 47, wherein said treatment comprises administration of a pharmaceutical.

49. The method of claim 48, wherein said pharmaceutical comprises one or more pharmaceuticals selected from the group consisting of a statin, a beta blocker, nitroglycerin or other nitrate, heparin, ACE inhibitor, angiotensin receptor blockers (ARB), aspirin and other anti-platelets, calcium channel blocker, and Ranolazine.

50. The method according to any one of claims 47-49, wherein said treatment is a treatment selected from the group consisting of angioplasty, percutaneous intervention (PCI) including implantation of a stent, and coronary bypass surgery.

51. A kit for performing a method of evaluating HDL function, said kit comprising:
a container containing AMPLEX® RED® or AMPLEX® ULTRARED®; and
a container containing one or more reagents for isolating HDL.

52. The kit of claim 51, wherein said one or more reagents for isolating HDL comprise a reagent selected from the group consisting of PEG, heparin MnCL₂, sodium phosphotungstate, dextran sulfate, and an antibody for immunoaffinity capture of HDL.

53. The kit of claim 51, wherein said one or more reagents for isolating HDL comprise an antibody for immmunoaffinity capture of HDL.

54. The kit of claim 53, wherein said antibody is attached to a solid support.

55. A method of screening for an agent that improves HDL function, said method comprising:
contacting HDL with one or more test agents; and
determining the HLD redox activity of said HDL according to the method of any one of claims 1-33, where a decrease in the HRA of said HDL, or the prevention of an increase in the HRA of said HDL indicates that said one or more test agents improve HDL function.

56. The method of claim 55, wherein said contacting is ex vivo.

57. The method of claim 55, wherein said contacting comprises administering said one or more test agents to a mammal.
